(19) United States
(12) Patent Application Publication
(10) Pub. No.: US 2009/0131551 A1
(43) Pub. Date: May 21, 2009

(54) POLYFUNCTIONAL COMPOUNDS AND USES AS IMPLANT MATERIALS

(76) Inventor: Dong Xie, Westfield, IN (US)

Correspondence Address: BARNES & THORNBURG LLP, 11 SOUTH MERIDIAN, INDIANAPOLIS, IN, 46204, US

(21) Appl. No.: 12/280,929

(22) PCT Filed: Feb. 27, 2007
(86) PCT No.: PCT/US07/62882

§ 371 Date: Dec. 4, 2008

Related U.S. Application Data
(60) Provisional application No. 60/777,827, filed on Mar. 1, 2006.

(51) Int. Cl. A61K 6/083; C07C 69/003; A61Q 11/00; C07C 229/04

(52) U.S. Cl. 523/115; 560/129; 560/171

(57) ABSTRACT

The synthesis and characterization of polymer core initiators are described herein. Polymer core initiators are used to prepare the polyfunctional prepolymers described herein, which may be optionally tethered. The polyfunctional prepolymers described herein are used to prepare cements, optionally with added co-monomers, for repairing and restoring tissues.

### GOVERNMENT RIGHTS

This invention was made at least in part with funding from the National Institute of Biomedical Imaging and Bioengineering with The National Institutes of Health Grant No. R01 EB03162-03; the United States Government may have certain rights in this invention.

### TECHNICAL FIELD

The invention described herein pertains to polymers. In particular, the invention described herein pertains to polymers that include polyfunctional core molecules. The polymers described herein may be useful as prosthetic implants.

### BACKGROUND

Glass-ionomer cements (GICs) are used as restorative materials in dentistry, as described by Smith D C. “Development of glass-ionomer cement systems” Biomaterials 1998; 19:467-478; Wilson A D, McLean J W. “Glass-ionomer cements” Chicago, Ill.: Quintessence Publ Co.; 1988; Davidson C L, Mjör I A. “Advances in glass-ionomer cements” Chicago, Ill.: Quintessence Publ Co.; 1999.; Wilson A D. “Resin-modified glass-ionomer cement” Int J Prosthodont 1990; 3:425-429. The success of these cements may be attributed to such properties including direct adhesion to tooth structures and base metals, as described by Hotz P, McLean J W, Sced I, Wilson A D. “The bonding of glass-ionomer cements to metal and tooth substrates” Br Dent J 1977; 142:41-47; Lacefield W R, Reindl M C, Retief D H. “Tensile bond strength of a glass-ionomer cement” J Prosthet Dent 1985; 53:194-198; anticariogenic properties due to release of fluoride, as described by Forsten, L. “Fluoride release from a glass-ionomer cement” Scand J Dent Res 1977; 85:503-4; thermal compatibility with tooth enamel and dentin due to low coefficients of thermal expansion similar to that of tooth structure and minimized microleakage at the tooth-enamel interface due to low shrinkage, as described by Craig R G. “Restorative Dental Materials” 10^{th} ed. St Louis, Mo.: Mosby-Year Book, Inc.; 1997; and low cytotoxicity, as described by Nicholson J W, Braybrook J H, Wasson E A. “The biocompatibility of glass-poly(alkenoate) glass-ionomer cements: a review” J Biomater Sci Polym Edn 1991; 2(4):277-285; Hume W R, Mount G J. “In vitro studies on the potential for pulpal cytotoxicity of glass-ionomer cements” J Dent Res 1988; 67(6):915-918. Each of the foregoing publications is incorporated herein by reference.

The setting and adhesion mechanisms of GICs to dental materials may arise from the acid-base reaction between calcium and/or aluminum cations released from or present on the surfaces of a reactive glass, and the carboxyl anions present on the polyacid. The polyacids used in the formation of GICs are generally linear polymers, synthesized via conventional free-radical polymerization. Illustrative polymer backbones of GICs are made from poly(acrylic acid) homopolymer, poly(acrylic acid-co-itaconic acid) or/and poly(acrylic acid-co-maleic acid) copolymers. Such GICs are often referred to as conventional glass-ionomer cements (CGICs) or self-cured GICs. However, some conventional GICs may be too brittle or have insufficient tensile and flexural strengths for some applications, and thus are useful only at certain low stress-bearing sites such as Class III and Class V cavities. Prior efforts to improve the mechanical strengths of CGICs have focused on changing the linear polymer backbone or matrix. Of two main strategies applied, the first is to incorporate hydrophobic pendent (meth)acrylate moieties onto the polyacid backbone of the CGIC to prepare a light-initiated or redox-initiated resin-modified GIC (RMGIC). Such modifications have been shown to improve tensile and flexural strengths as well as handling properties. A second strategy is to increase the molecular weight (MW) of the polyacid polymer, by either introducing amino acid derivatives or N-vinylpyrrolidone. Such modifications have also shown enhanced mechanical strengths. However, the working properties of those higher molecular weight polymers were decreased, due in part to the increased solution viscosity, because of the corresponding higher degree of strong chain entanglements that may be formed in these high MW linear polyacids. Therefore, a continuing need remains for providing implant materials that have both the desirable workability properties and the desirable mechanical properties for certain applications, including for implantation at high-stress sites.

### SUMMARY OF THE INVENTION

It has been discovered that polymers that include polyfunctional core molecules, such as star, hyperbranched, spherical, or dendritic shaped molecules, are useful as prostheses or implants in various tissue repair and/or restoration procedures. It has also been discovered that the monomers used to make such polymers, including those described herein may demonstrate low solution or melt viscosity, thus providing improved workability characteristics. Without being bound by theory, it is suggested that such polyfunctional core molecules, and the prepolymer oligomers and polymers prepared therefrom may behave like solutions of spheres and therefore may exhibit fewer chain entanglements. It is further suggested that limiting chain entanglements in such prepolymer oligomers and/or polymers may be beneficial to polymer processing, as described by Bahadur P, Sastry N V. “Principles of Polymer Science” Boca Raton, Fla.: CRC press; 2002; Huang C F, Lee H F, Kuo S W, Xu H, Chang F C. “Star polymers via atom transfer radical polymerization from adamantine-based cores” Polymer 2004; 45:2261-2269. Further, it has been discovered that the molecular weights of such monomers and polymers prepared from the polyfunctional core molecules described herein may be increased without a corresponding increase, or with proportionally less of a corresponding increase, in the viscosity of such polymers, and solutions thereof. It has also been discovered that cements may be prepared from such monomers and polymers prepared from the polyfunctional core molecules, and those cements may have improved mechanical strength properties over conventional cements.

In one illustrative embodiment of the invention, polymers and prepolymer oligomers are described herein. In one aspect, such prepolymer oligomers are polyfunctional core molecules that may be used to initiate the preparation of a polymer. Illustrative polymer core initiators are described that include a polyfunctional core molecule. As used herein, polyfunctional core refers to molecules that have a plurality of functional groups that may be optionally used to initiate polymer chains, or which may be modified with oligomers or other prepolymers, each of which may be optionally used to initiate polymer chains.

In one illustrative embodiment, initiators are described that are prepared from a polyfunctional core molecule, where each of the functional groups present on the polyfunctional core molecule is covalently attached to another molecule that includes a functional group capable of participating in a polymerization reaction with a plurality of acrylates. In another embodiment, polyfunctional prepolymers are described herein. Such polyfunctional prepolymers are prepared from the polymer core initiators by polymerizing a plurality of acrylates. In another embodiment, polyfunctional prepolymers are further functionalized by tethering one or more acryloyl substituted groups as amides and/or esters of the acrylates. In another embodiment, cements useful in the repair and/or restoration of tissues are described. Such cements may be prepared directly from the polyfunctional prepolymers and/or tethered polyfunctional prepolymers described herein. In one variation, the cements may be prepared by co-polymerization of one or more co-monomers and the polyfunctional prepolymers and/or tethered polyfunctional prepolymers described herein. In another variation, the cements may be prepared by adding inorganic fillers, such as glasses, ceramics, biological tissues, and the like, to the polymerizing polyfunctional prepolymers and/or tethered polyfunctional prepolymers, with the optional inclusion of other co-monomers.

In another illustrative embodiment of the invention, processes for preparing polymer core initiators, polyfunctional prepolymers, and tethered polyfunctional prepolymers are described herein, including polymerization performed using living free-radical polymerization technologies such as atom-transfer radical polymerization (ATRP). Additional synthetic details are described by Matyjaszewski K, Xia J. “Atom transfer radical polymerization” Chem Rev 2001; 101:2921-2990. In another embodiment, processes for preparing cements and cement compositions are described herein. The polyfunctional prepolymers, and tethered polyfunctional prepolymers, optionally in the presence of one or more co-monomers, are curable by radiation, heat, and/or radical initiation.

In another embodiment, processes for preparing the polyfunctional core initiators, polyfunctional prepolymers, and implant polymers are described herein

In another illustrative embodiment of the invention, methods for using the polyfunctional core initiators, polyfunctional prepolymers, and implant polymers described herein as cements for the repair and/or restoration of tissue are described herein.

### BRIEF DESCRIPTION OF THE DRAWINGS

FIG. 1 shows FT-IR spectra for initiators and polymers: (a) BIBB and 4-arm BIBB initiator; (b) t-BA, 4-arm poly(t-BA), 4-arm poly(AA), IEM-tethered 4-arm poly(AA) and GM-tethered 4-arm poly(AA).

FIG. 2 shows ¹H NMR spectra for initiators and polymers: 4-arm BIBB initiator; 4-arm PAA, IEM-tethered 4-arm PAA and GM-tethered 4-arm PAA.

FIGS. 3(a) and 3(b) show the conversion and kinetic plot of the 4-arm poly(t-BA) derived from the FT-IR absorbance spectra. (a): Conversion vs. time curve; (b): First-order kinetic plot of ln([M]ₒ/[M]) vs. time. The 4-arm poly(t-BA) was prepared in dioxane via ATRP in the presence of the 4-arm BIBB, CuBr, and PMDETA.

FIG. 4 shows the yield compressive strength (YCS), ultimate compressive strength (UCS), and modulus (M) of illustrative self-cured Examples A-C, compared to linear Example D: The compositions of Examples A-D are shown in Table 1; where the P/L ratio=2.7. The polymer solution was prepared by mixing a PAA with distilled water (1:1, by weight). Specimens were conditioned in distilled water at 37° C. for 24 h.

FIG. 5 shows the compressive strength (CS) and diametral tensile strength (DTS) of illustrative light-cured cements: Examples E-L refer to compositions as defined in Table 2; where Mₙ of each of the polymers=18,066 Daltons; Filler=Fuji II LC; P/L ratio=2.7. Specimens were conditioned in distilled water at 37° C. for 24 h.

FIG. 6a shows the CS, DTS, and flexural strength (FS) of two selected illustrative cements described herein compared to Fuji II LC cement. For the illustrative cements described herein, MW of the polymer=18,066; Filler=Fuji II LC; P/L ratio=2.7. For Fuji II LC, P/L ratio=3.2. Specimens were conditioned in distilled water at 37° C. for 24 h.

FIG. 6b shows the CS, DTS and FS of Example M (EXPGIC) compared to Fuji II, Fuji II LC and Vitremer. For Example M: MW of the polymer=18,066; filler=Fuji II LC; P/L ratio=2.7. For Fuji II, Fuji II LC and Vitremer, P/L ratio=2.7, 3.2, and 2.5, respectively. Specimens were conditioned in distilled water at 37° C. for 24 h.

FIGS. 7(a) and 7(b) show the conversion and kinetic plot of the 4-arm poly(t-BA) derived from the FT-IR absorbance spectra: (a) Conversion vs. time curve; (b) First-order kinetic plot of ln([M]ₒ/[M]) vs. time. The 4-arm poly(t-BA) was prepared in dioxane via ATRP in the presence of the 4-arm BIBB, CuBr and PMDETA

FIG. 8 shows the CS and DTS of the light-cured GM-tethered PAA-constructed Examples B-I: P/W ratio and grafting ratio are described in Table 2; Filler=Fuji II LC; P/L ratio=2.7. Specimens were conditioned in distilled water at 37° C. for 24 h prior to testing.

FIG. 9 shows the CS, DTS and FS of illustrative cements described herein compared to Fuji II LC. For the illustrative cements, GM grafting ratio=70%; P/W ratio=75/25; P/L ratio=2.7; For Fuji II LC, P/L ratio=3.2. Specimens were conditioned in distilled water at 37° C. for 24 h.

FIG. 10 shows the change in CS for Example M (EXPGIC), Fuji II, Fuji II LC and Vitremer in the course of aging in water. The h, d and w represent hour, day and week, respectively. Specimens were conditioned in distilled water at 37° C. prior to testing.

FIG. 11 shows the cell viability comparison after culturing for 3 days with the eluates from selected cements. Eluates were obtained from the 3-day and 7-day incubation at a concentration of 80%. EXPGIC is Example M; NC is the negative control.

FIGS. 12(a) and 12(b) show cell viability (% survival) vs. cement eluate concentration: (a) Eluates obtained from a 3-day incubation; (b) Eluates obtained from a 7-day incubation. The cells were incubated with the medium containing different concentrations of the eluates at 37° C. for 3 days before MTT testing. EXPGIC is Example M; NC is the negative control.

FIGS. 13(a) to 13(e) show cell morphology and density (200× magnification): (a) negative control; (b) Example M; (c) Fuji II; (d) Fuji II LC; (e) Vitremer. Cell morphology photomicrographs were obtained after the cells incubated with the 7-day eluates for 3 days.

### DETAILED DESCRIPTION

Polymer core initiators are described herein. Such polymer core initiators may include from 3 to about 12 functional groups for polymerization. In one embodiment, the polymer core initiators may include 3, 4, 5, or 6 functional groups for polymerization. In one embodiment, the polymer core initiators are dendrimeric and may include from about 8 to about 12, or from about 10 to about 12 functional groups for polymerization. The functional groups may be leaving groups or electrophiles such as halo, alkoxy, acyloxy, sulfonyloxy, and the like, nucleophiles such as hydroxy, amino, carboxy, and the like, or radical initiators such as halo, stannyl, and the like. In one embodiment, the polymer core initiators are prepared as esters from polyhydroxy compounds and carboxylic acids. Illustratively, the polyhydroxy compounds are poly(hydroxyalkyl) compounds including, but not limited to, tetramethylol propane (TMP), pentaerythritol (PE), dipentaerythritol (DPE), and the like. Illustratively, the carboxylic acids are omega halo alkanoic acids, such as chloroacetic acid, 2-bromopropanoic acid, 3-iodopropanoic acid, 2-bromo-2-methylpropanoic acid, and the like.

Illustratively, the polymer core initiators are compounds of the formulae (I):

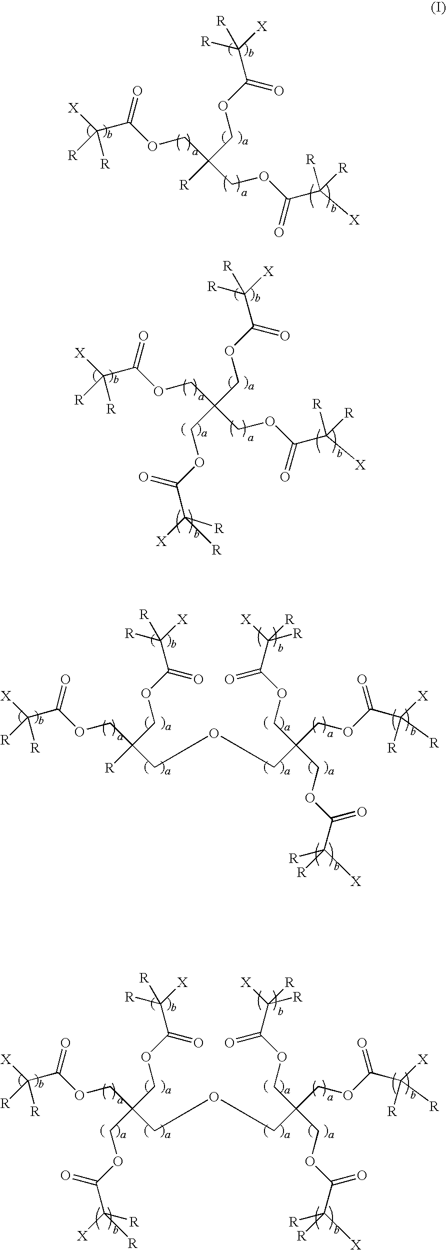

wherein in each instance, R is hydrogen or an independently selected alkyl group, a is an independently selected integer from 1 to about 4, b is an independently selected integer from 1 to about 4, and X is an independently selected leaving group, such as halo, acyloxy, sulfonyloxy, and the like.

In another embodiment, the polymer core initiators described herein are compounds of formulae (I) where a and b are each independently selected from 1 and 2. In another embodiment, the polymer core initiators described herein are compounds of formulae (I) where R is in each case independently selected from hydrogen or lower alkyl, such as methyl, ethyl, and the like. In another embodiment, the polymer core initiators described herein are compounds of formulae (I) where X is halo.

Polyfunctional prepolymers are described herein. In one embodiment, the polyfunctional prepolymers are polymer core initiators further functionalized with poly(acrylic acid)s (PAA)s. It to be understood that as used herein, the term poly(acrylic acid) refers both to substituted and unsubstituted acrylic acids. Illustratively, PAAs include, but are not limited to, homo and co-polymers of acrylic acid, methacrylic acid, crotonic acid, maleic acid, fumaric acid, itaconic acid, citraconic acid, mesaconic acid, and the like. In addition, it is to be understood that as used herein, the acrylic acid starting materials that are used to prepare the PAAs described herein may be esters, amides, or acid salts. Illustratively, acrylic acid starting materials include methyl esters, ethyl esters, tert-butyl esters and the like. Further, acrylic acid starting materials include amides, alkylamides, dialkylamides, dipeptides, and the like. Further, acrylic acid starting materials include monovalent and polyvalent cationic salts such as lithium, sodium, potassium, cesium, calcium, magnesium, and the like.

Illustratively, the polyfunctional prepolymer is a compound of the formulae (II):

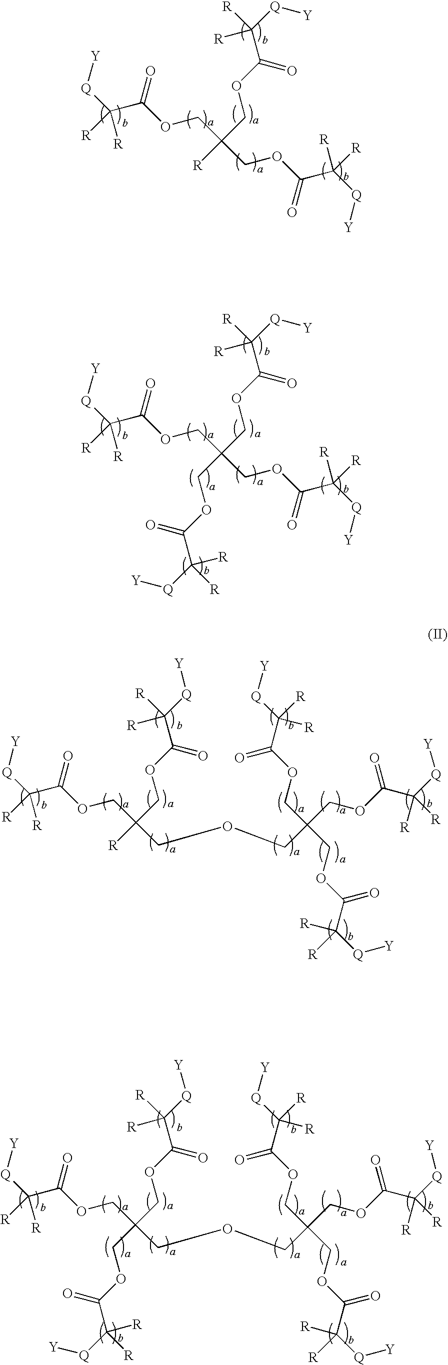

wherein in each instance R is hydrogen or an independently selected alkyl group; a is an independently selected integer from 1 to about 4; b is an independently selected integer from 1 to about 4; Q is an independently selected polymer, which may be a statistically distributed polymer, a random polymer, a grafting co-polymer, block copolymer, and the like, of one or more acrylic acids, or ester, amide, or salt derivatives thereof, and Y is an independently selected leaving group, such as halo, acyloxy, sulfonyloxy, and the like.

In another embodiment, the polyfunctional prepolymers described herein are compounds of formulae (II) where a and b are each independently selected from 1 and 2. In another embodiment, the polyfunctional prepolymer described herein are compounds of formulae (II) where R is in each case independently selected from hydrogen or lower alkyl, such as C₁-C₄ alkyl, methyl, ethyl, and the like. In another embodiment, the polyfunctional prepolymer described herein are compounds of formulae (II) where Y is halo. In another embodiment, the polyfunctional prepolymer described herein are compounds of formulae (II) where Q is a homopolymer or copolymer of acrylic acid, methacrylic acid, maleic acid, and/or itaconic acid, or one or more carboxylic acid derivatives thereof. In another embodiment, the polyfunctional prepolymer described herein are compounds of formulae (II) where Q is a homopolymer or copolymer of acrylic acid and/or methacrylic acid, or one or more carboxylic acid derivatives thereof. In one variation, Q is a homopolymer or copolymer of acrylic acid and/or methacrylic acid amides. In another variation, Q is a homopolymer or copolymer of acrylic acid and/or methacrylic acid esters.

Tethered polyfunctional prepolymers are described herein. In one embodiment, the tethered polyfunctional prepolymers are polyfunctional prepolymers further functionalized as acryloyloxy substituted alkyl esters or acryloyloxy substituted alkyl amides. In another embodiment, the tethered polyfunctional prepolymers are polyfunctional prepolymers further functionalized as acryloylamino substituted alkyl esters or acryloylamino substituted alkyl amides. As described herein, acryloyl is understood to refer to substituted and unsubstituted acryloyls. Illustratively, acryloyls include, but are not limited to, acryloyl, methacryloyl, crotonoyl, maleoyl, fumaroyl, itaconoyl, citraconoyl, mesaconoyl, and the like. In one embodiment, the acryloyl is curable with radiation. In another embodiment, the acryloyl is curable under radical conditions, such as in the presence of heat and/or a radical initiator. In another embodiment, the acryloyl is a methacryloyl. In another embodiment, the substituted alkyl esters or substituted alkyl amides tethered to the polyfunctional prepolymers are prepared from acryloyloxy and acryloylamino alkylisocyanates, alkylepoxides, alkanols, alkylcarboxylic acids, and derivatives thereof, and the like.

Illustratively, the tethered polyfunctional prepolymer is a compound of the formulae (III):

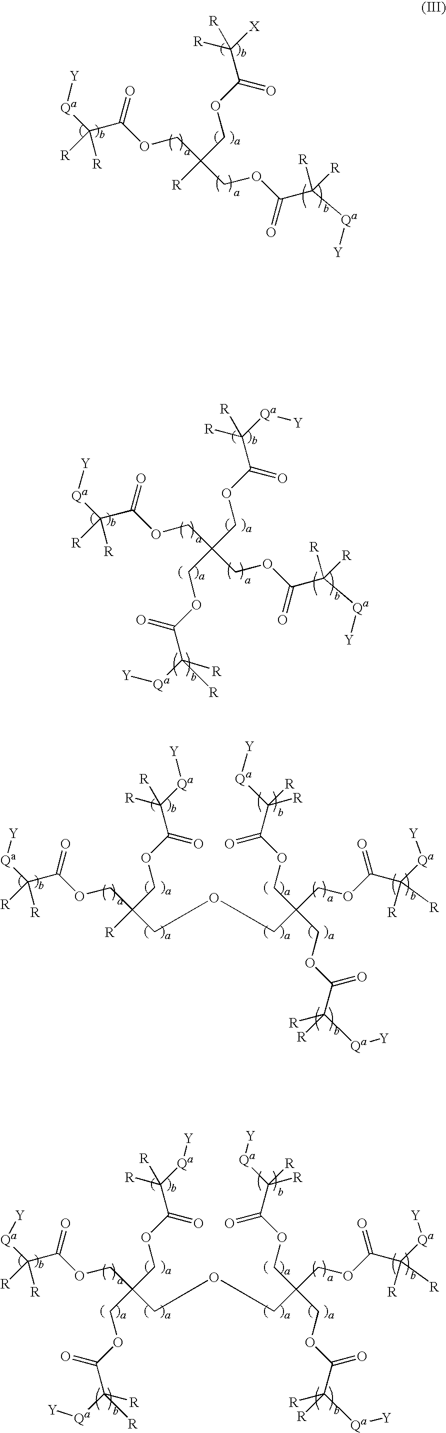

wherein in each instance R is hydrogen or an independently selected alkyl group; a is an independently selected integer from 1 to about 4; b is an independently selected integer from 1 to about 4; Q^{a} is an independently selected polymer, which may be a statistically distributed polymer, a random polymer, a grafting co-polymer, block copolymer, and the like, of one or more acrylic acids, or ester, amide, or salt derivatives thereof; and Y is an independently selected leaving group;

providing that at least one of the acrylic acids forming the polymer Q^{a} is an ester or amide of an alcohol or amine each independently selected from the group consisting of acryloyloxyalkanols, acryloyloxyalkylamines, acryloylaminoalkanols, and acryloylaminoalkylamines, each of which is optionally substituted, such as with alkyl, hydroxy, halo, carboxyl, and the like.

In another embodiment, the tethered polyfunctional prepolymers described herein are compounds of formulae (III) where a and b are each independently selected from 1 and 2. In another embodiment, the polyfunctional prepolymer described herein are compounds of formulae (III) where R is in each case independently selected from hydrogen or lower alkyl, such as methyl, ethyl, and the like. In another embodiment, the polyfunctional prepolymer described herein are compounds of formulae (III) where Y is halo. In another embodiment, the polyfunctional prepolymer described herein are compounds of formulae (III) where Q^{a} is a homopolymer or copolymer of acrylic acid, methacrylic acid, maleic acid, and/or itaconic acid, or one or more carboxylic acid derivatives thereof. In another embodiment, the polyfunctional prepolymer described herein are compounds of formulae (III) where Q^{a} is a homopolymer or copolymer of acrylic acid and/or methacrylic acid, or one or more carboxylic acid derivatives thereof. In one variation, Q^{a} is a homopolymer or copolymer of one or more acrylic acids, acrylic acid amides, methacrylic acids, and/or methacrylic acid amides. In another variation, Q^{a} is a homopolymer or copolymer of one or more acrylic acids, acrylic acid esters, methacrylic acids, and/or methacrylic acid esters. In another variation, Q^{a} includes a plurality of acryloyloxyalkanols, such as acryloyl and/or methacryloyl ethanol. In another variation, Q^{a} includes a plurality of acryloyloxyalkanols, such as acryloyl and/or methacryloyl glycerols. In another variation, Q^{a} includes a plurality of acryloyloxyalkylamines, such as acryloyl and/or methacryloyl ethylamine. In another variation, Q^{a} includes a plurality of acryloyloxyalkylamines, such as acryloyl and/or methacryloyl ethylamine.

Co-monomers of the polyfunctional prepolymers and tethered polyfunctional prepolymers are described herein. In one embodiment, the co-monomer is a hydroxy, amino, and/or carboxylic acid substituted alkyl amide or ester of an acrylate. As described herein, acrylate is understood to refer to substituted and unsubstituted acrylates. Illustratively, acrylates include, but are not limited to, acrylate, methacrylate, crotonate, maleate, fumarate, itaconate, citraconate, mesaconate, and the like. In variations of the embodiments described herein, such co-monomers are optionally added to polyfunctional prepolymers and/or tethered polyfunctional prepolymers during curing to prepare polymers. It is appreciated that the addition of one or more co-monomers may increase the water solubility, hydrophilicity, and/or solvation of the polymers prepared from polyfunctional prepolymers and/or tethered polyfunctional prepolymers. In addition, it is further appreciated that the addition of one or more co-monomers may increase the homogeneity of composites prepared from polyfunctional prepolymers and/or tethered polyfunctional prepolymers, and fillers, such as glasses, ceramics, other inorganic materials, and the like. In one embodiment, the co-monomer is curable with radiation. In another embodiment, the co-monomer is curable under radical conditions, such as in the presence of heat and/or a radical initiator. Illustratively, the co-monomer is a hydroxyalkyl ester of methacrylate, or a carboxylalkylamide of methacrylate.

In another embodiment, GICs prepared from polyfunctional prepolymers and/or tethered polyfunctional prepolymers that do not include added co-monomers are described herein. It is appreciated that light-cured RMGICs described herein may have certain advantageous chemical and mechanical features, such as reduced moisture sensitivity, improved mechanical strengths, extended working time, ease of clinical handling, and the like. The advantages of such chemical and mechanical features are described by D.C. Smith, “Development of glass-ionomer cement systems” Biomaterials 19 (1998) 467-478; A. D. Wilson, “Resin-modified glass-ionomer cement” Int. J. Prosthodont. 3 (1990) 425-429. It is also appreciated that light-cured RMGICs described herein may exhibit improved biocompatibility. Such advantages are described by J. W. Nicholson, J. H. Braybrook, and E. A. Wasson, “The biocompatibility of glass-poly(alkenoate) glass-ionomer cements: a review” J. Biomater. Sci. Polym. Edn. 2(4) (1991) 277-285; W. R. Hume and G. J. Mount, “In vitro studies on the potential for pulpal cytotoxicity of glass-ionomer cements” J. Dent. Res. 67(6) (1988) 915-918. Illustratively, it has been reported that RMGICs may generally be less biocompatible than CGICs, as described by C. A. de Souza Costa, J. Hebling, F. Garcia-Godoy, and C. T. Hanks, “In vitro cytotoxicity of five glass-ionomer cements” Biomaterials 24 (2003) 3853-3858; G. Leyhausen, M. Abtahi, M. Karbakhsch, A. Sapotnick, and W. Geustsen, “Biocompatibility of various light-curing and one conventional glass-ionomer cements” Biomaterials, 19 (1998) 559-564. It has been suggested that one source of decreased biocompatibility may be attributed to the presence of 2-hydroxyethyl methacrylate (HEMA) in the co-monomer added to the conventional GIC. Unfortunately, it is also understood that the addition of HEMA may be responsible for the observed enhancement in water solubility of the methacrylate-containing polyacids. It is appreciated that residual HEMA from incomplete polymerization may leach from RMGICs such as Vitremer and Compoglass, and exhibit cytotoxicity after contacting the dental pulp tissue and osteoblasts, further explaining why CGICs show less cytotoxicity to dental pulp or the other tissues. Each of the disclosures of the cited publications are incorporated herein by reference.

It is also suggested that conventional RMGICs require low MW amphiphilic molecules like HEMA. Accordingly, described herein are polyfunctional prepolymers tethered to amphiphilic methacrylate functionalities. It is further suggested that such tethering onto the polyfunctional prepolymers may substitute for the HEMA-based hydrophobic methacrylate moieties incorporated into conventional RMGICs.

Syntheses of polymer core initiators are described herein. Also described herein are syntheses of polyfunctional prepolymers. Also described herein are syntheses of tethered polyfunctional prepolymers. It is understood that conventional radical initiated polymerization of some or all polyfunctional prepolymers may be difficult impossible. Accordingly, described herein are alternate syntheses of such compounds using atom-transfer radical polymerization (ATRP) processes and techniques.

In another embodiment, 4-arm PAA polyfunctional prepolymers are synthesized using ATRP. The 4-arm PAAs may also be tethered with various substituted acrylate and methacrylate esters, such as 2-isocyanatoethyl methacrylate (IEM), glycidyl methacrylate (GM), 2-hydroxyethyl methacrylate (HEMA), methacryloyl beta-alanine (MBA), and the like. The polyfunctional prepolymers and tethered polyfunctional prepolymers described herein may also be formulated with co-monomers such as HEMA and/or MBA, in addition to water, and various optional polymerization initiators. In one variation, the polymerization of the polyfunctional prepolymers and tethered polyfunctional prepolymers is initiated by radiation. In another variation, the polymerization of the polyfunctional prepolymers and tethered polyfunctional prepolymers, with the optional addition of one or more co-monomers, is performed in the presence of one or more ceramic or glass fillers, including but not limited to various forms of hydroxyapatite, commercially available ceramics, including FUJI II LC filler, and the like.

Light-cured, self-cured, and radical cured glass-ionomer cements (GICs) are described herein. In one embodiment, the GIC is prepared from one or more polyfunctional prepolymers. In another embodiment, GIC is prepared from one or more tethered polyfunctional prepolymers. In another embodiment, the GIC is prepared from one or more polyfunctional prepolymers and one or more tethered polyfunctional prepolymers. In another embodiment, the GIC is prepared as described herein in the presence of one or more co-monomers. In one variation, the GIC is prepared from one or more tethered polyfunctional prepolymers and one or more co-monomers. In another variation GIC is prepared from one or more tethered polyfunctional prepolymers in the absence of any added co-monomers.

GICs are described herein that exhibit improved mechanical properties, including improved mechanical strengths. In one embodiment, the cements described herein are evaluated for their mechanical properties. Mechanical properties include various mechanical strength parameters, including but not limited to compressive strength (CS), tensile strength (TS), toughness, modulus (M), and the like.

It is appreciated that polyfunctional prepolymers, tethered polyfunctional prepolymers, and cements described herein may exhibit improved physical properties, workability properties, and mechanical properties than conventional prepolymers and cements. In one aspect, polyfunctional prepolymers and/or tethered polyfunctional prepolymers described herein have a lower viscosity as compared to the corresponding linear counterpart, or conventional prepolymer.

In another aspect, cements prepared from polyfunctional prepolymers and/or tethered polyfunctional prepolymers described herein show higher mechanical strengths than corresponding conventional cements. For example, cements (LCGICs) prepared from both IEM-tethered PAAs and GM-tethered 4-arm PAAs show higher mechanical strengths than the cements prepared from the corresponding linear prepolymers. In addition, it is appreciated that the cements prepared from IEM-tethered PAAs may show higher CS and DTS than the corresponding cements prepared from GM-tethered PAAs. In addition, it is appreciated that the cements prepared with MBA co-monomer may exhibit higher CS than the corresponding cements prepared with UEMA. Without being bound by theory, it is suggested that the MBA-containing PAA cement may exhibit higher CS than the corresponding HEMA-containing PAA cements due to salt-bridge contributions between the MBA and the filler or ceramic added to the composite. The IEM-tethered cements may show higher mechanical strengths than corresponding GM-tethered cements, possibly due to a hydrophobicity difference between the two corresponding polymers.

In another embodiment, the effects of grafting ratio, polymer/water (P/W) ratio, filler powder/polymer liquid (P/L) ratio, and aging on strengths are described for LCGICs prepared from polyfunctional prepolymers and/or tethered polyfunctional prepolymers that are not polymerized or cured with any co-monomer. In one embodiment, the 4-arm PAA polymer may exhibit a lower viscosity compared to the corresponding linear counterpart synthesized via conventional free-radical polymerization. For such monomer-free cements, increasing P/W ratio may increase both CS and DTS; increasing grafting ratio may increase CS; and increasing P/L ratio may increase CS. Also for such monomer-free cements, aging may allow the ultimate CS (MPa) to increase over time. It is appreciated that monomer-free LCGICs may have the advantage of lower cytotoxicity to dental tissue due to the absence of monomers, such as HEMA, that may remain in some polymerized cements and leach into tissues.

In another embodiment, kits are described herein. The kit may include one or more polyfunctional prepolymers and/or one or more tethered polyfunctional prepolymers. The kit may also include other formulating materials, including but not limited to co-monomers, initiators, and fillers.

In another embodiment, methods for repairing, and/or restoring tissue are described herein. Illustrative tissues that may be repaired or restored include but are not limited to dental tissues, bone tissues, and cartilage tissues. The polyfunctional prepolymers, tethered polyfunctional prepolymers, and cements described herein may be used as replacement materials for conventional GICs. In one embodiment of the methods, a curable composition including one or more of the polyfunctional prepolymers and/or one or more tethered polyfunctional prepolymers is placed in the defect, and cured. Curing may take place by initiating with radiation, and/or a chemical reagent, such as a radical initiator. The polyfunctional prepolymers, tethered polyfunctional prepolymers, and cements described herein may also be used in conjunction with other prosthetic materials in the repair or restoration of the tissue.

### EXAMPLES

The following abbreviations are used herein: Trimethylolpropane (TMP), Pentaerythritol (PE), triethylamine (TEA), Dipentaerythritol (DPA), 2-bromoisobutyryl bromide (BIBB), Cuprous bromide (CuBr), N,N,N′,N′,N″-pentamethyldiethylenetriamine (PMDETA), dl-camphoroquinone (CQ), diphenyliodonium chloride (DC), 2,2′-azobisisobutyronitrile (AIBN), dibutyltin dlaurate (DBTL), triphenylstibine (TPS), pyridine (C₆H₅N), tert-butyl acrylate (t-BA), methacryloyl chloride, beta-alanine (BA), 2-hydroxyethyl methacrylate (HEMA), 2-isocyanatoethyl methacrylate (IEM), glycidyl methacrylate (GM), anhydrous magnesium sulfate (MgSO₄), sodium hydroxide (NaOH), hydrochloric acid (HCl, 37%), diethyl ether (Et₂O), tetrahydrofuran (THF), methanol (MeOH), deuterated methyl sulfoxide (DMSO-d₆), and ethyl acetate (EtOAc). Each reagent was used as received from commercial suppliers. GC FUJI II and GC FUJI II LC glass powders were supplied by GC America Inc (Alsip, Ill.).

### Example

Synthesis of the 4-arm pentaerythritol tetrakis(2-bromoisobutyrate) initiator. To a reactor charged with 100 ml (0.72 mole) of TEA, 15 g (0.11 mole) of pentaerythritol and 200 ml of THF, a mixture of 100 ml (0.81 mole) of BIBB in 25 ml of THF was added dropwise with stirring at room temperature. After addition was completed, additional one hour was added to complete the reaction. The solution was washed with 5% NaOH and 1% HCl and then extracted with ethyl acetate. The extract was dried with anhydrous MgSO₄, concentrated in vacuo and crystallized. The final product was re-crystallized from diethyl ether. The schematic diagram for the 4-arm initiator synthesis is shown in Scheme 1a. Additional synthetic details are described by Wang X, Zhang H, Zhong G, Wang X. “Synthesis and characterization of four-armed star mesogen-jacketed liquid crystal polymer” Polymer 2004; 45(11):3637-3642.

Schemes 1(a)-1(c) describe illustrative syntheses: (a) Synthesis of the 4-arm PAA: (1) Synthesis of the 4-arm BIBB initiator; (2) Synthesis of the 4-arm poly(t-BA) via ATRP; and (3) Hydrolysis of the 4-arm poly(t-BA); (b) Tethering either IEM or GM onto the 4-arm PAA; (c) Chemical structures of MBA and HEMA.

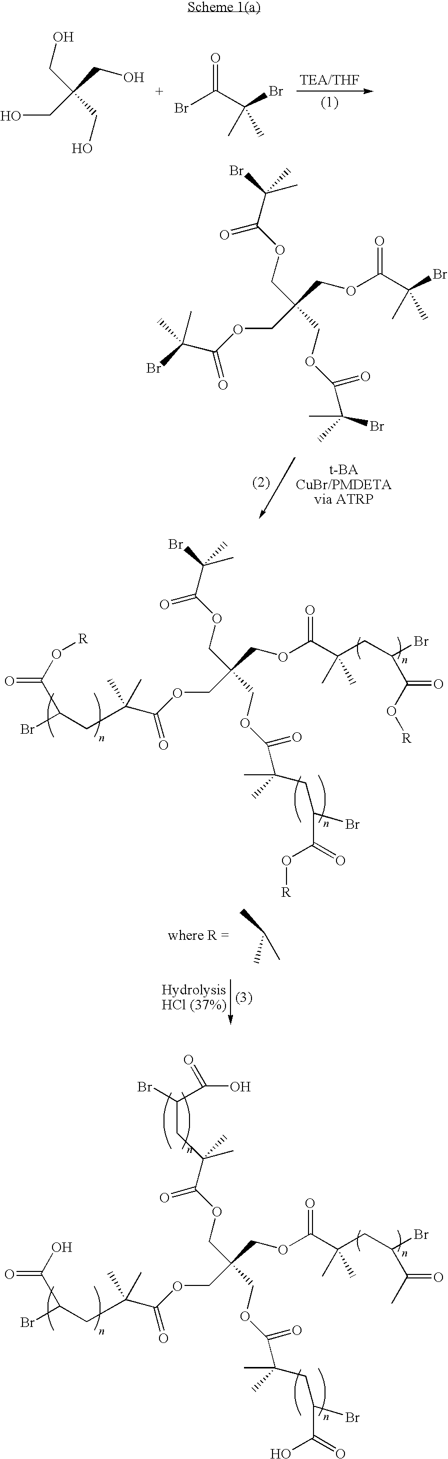

In Scheme 1 (a), n is in each instance an independently selected integer, which when selected collectively corresponds to an average molecular weight (Mₙ) of the polymer in the range from about 1,000 to about 50,000. Illustratively, the integers n are values that collectively correspond to an average molecular weight (Mₙ) of the polymer in the range from about 5,000 to about 30,000, or in the range from about 9,000 to about 22,000. In addition, it is to be understood that the preparation described in Scheme 1(b) may be used for other polymer core initiators and for other acrylates, by changing the starting compounds to those desired.

### Example

Synthesis of the 4-arm poly(acrylic acid) via ATRP. To a flask containing dioxane (5.0 g or 0.056 mole), 4-arm initiator (1% by mole), PMDETA (3%, ligand) and t-BA (5.0 g or 0.04 mole) were charged. The CuBr (3%) was incorporated under N₂ purging after the above solution was degassed and nitrogen-purged by three freeze-thaw cycles. The solution was then heated to 120° C. to initiate the ATRP. FT-IR was used to monitor the reaction. After the polymerization was completed, the poly(t-BA) polymer was precipitated from water. CuBr and PMDETA were removed by re-precipitated from dioxane/water. The colorless polymer was then hydrolyzed in a mixed solvent of dioxane and HCl (37%) (dioxane/HCl=⅓) under refluxed condition for 6-18 h, depending on the molecular weight of the polymer. The hydrolyzed poly(acrylic acid) was dialyzed against water until the pH in water became neutral. The purified 4-arm poly(acrylic acid) (PAA) was obtained after freeze-dried. The reaction scheme for PAA synthesis via ATRP is described in Scheme 1a. Three 4-arm PAA polymers with the same feed t-BA were synthesized at the initiator concentration of 0.5, 1.0, and 1.5%, respectively. Additional synthetic details are described by Ibrahim K, Lofgren B, Seppala J. “Synthesis of tertiary-butyl acrylate polymers and preparation of diblock copolymers using atom transfer radical polymerization” Eur Polym J 2003; 39:2005-2010; Davis K A, Charleux B, Matyjaszewski K. “Preparation of block copolymers of polystyrene and poly(t-butyl acrylate) of various molecular weights and architectures by atom transfer radical polymerization” J Polym Sci A Polym Chem 2000; 38:2274-2283.

### Example

Synthesis of the IEM-tethered 4-arm PAA. To a three-neck flask containing PAA (4.1 g or 0.057 mole), THF (18 ml), BHT (0.1%, by weight), TPS (0.1%) and DBTL (2%), a mixture of IEM (3.1 g or 0.02 mole for 35% grafting or 4.4 g or 0.029 mole for 50% grafting) and 3.7 ml of THF was added dropwise at 40° C. under a nitrogen blanket. Fourier transform-infrared (FT-IR) spectroscopy was used to monitor the reaction. The polymer tethered with IEM was recovered by precipitation from diethyl ether, followed by drying in a vacuum oven at 23° C. The scheme for synthesis of the IEM-tethered PAA is described in Scheme 1b. Additional synthetic details are described by Xie D, Chung I-D, Wu W, Lemons J, Puckett A, Mays J. “An amino acid modified and non-HEMA containing glass-ionomer cement” Biomaterials 2004; 25(10): 1825-1830.

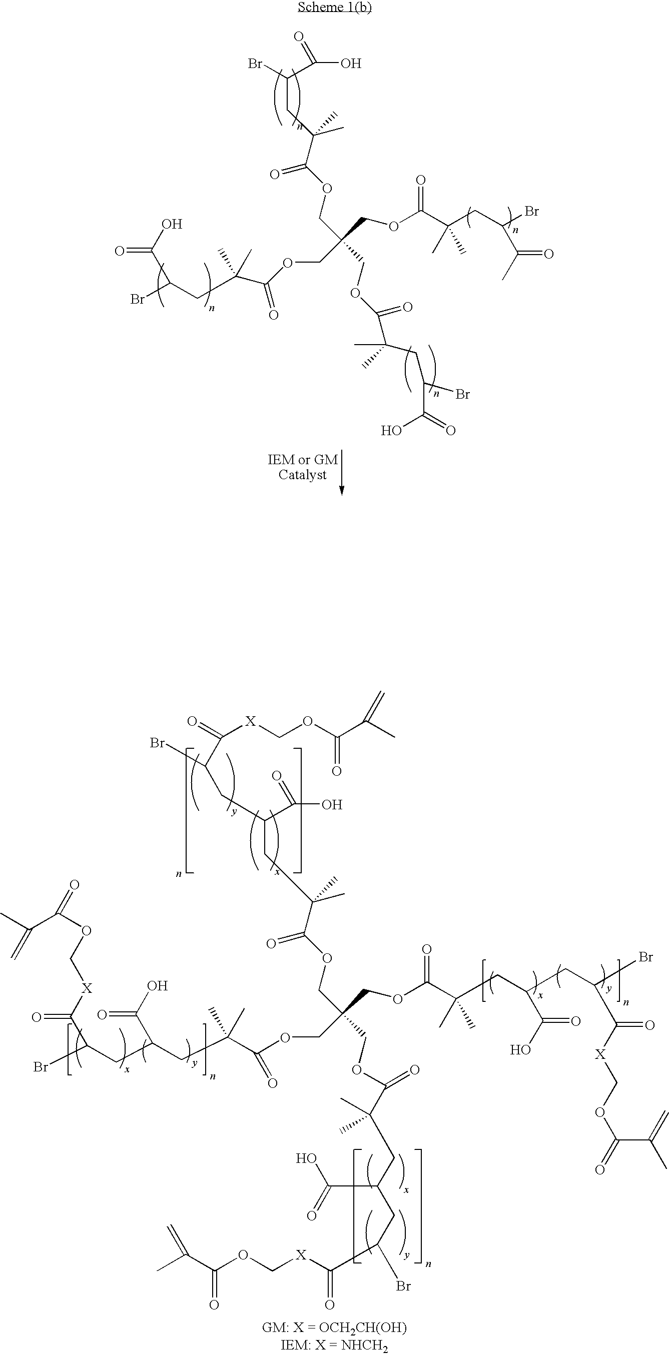

In Scheme 1(b), n is in each instance an independently selected integer, which when selected collectively correspond to an average molecular weight (Mₙ) of the polymer in the range from about 1,000 to about 50,000. Illustratively, the integers n are values that collectively correspond to an average molecular weight (Mₙ) of the polymer in the range from about 5,000 to about 30,000, or in the range from about 9,000 to about 22,000. Also in Scheme 1(b), x and y are integers, each of which is in each instance independently selected. It is therefore to be understood that the structures shown in Scheme 1(b) correspond to a variety of arrangements of the PAA and tethered PAA fragments. In one illustrative aspect, the values of each x, y, and n are such that a random polymeric chain results, or a statistically distributed polymeric chain results, where for example, the values of x and y in each case are small, such as less than 10, or less than 5. In another illustrative aspect, the values of each x, y, and n are such that the PAA and tethered PAA fragments form a graft polymer or block copolymer, where for example, the values of x and y in each case are large, such as greater than 10, or greater than 20. In another illustrative aspect, the values of each x, y, and n are diverse such that the PAA and tethered PAA fragments form random sections adjacent to block copolymeric sections. In addition, it is to be understood that the preparation described in Scheme 1(b) may be used for other polymer core initiators, for other acrylates, and for other tethering molecules by changing the starting compounds to those desired. It is therefore further appreciated that the nature of these numerous possible polymeric chain arrangements will vary with the selection of the polymer core initiators, the acrylates, and the tethering molecules.

### Example

Synthesis of the GM-tethered 4-arm PAA. To a three-neck flask containing PAA (4.1 g or 0.057 mole), THF (18 ml) and BHT (0.5%, by weight), a mixture of GM (2.8 g or 0.02 mole for 35% grafting or 4.0 g or 0.029 mole for 50% grafting), THF (21 ml), and pyridine (1% of GM, by weight) was added dropwise. Under a nitrogen blanket, the reaction was initiated and run at 60° C. for 5 h and then kept at room temperature overnight. FT-IR spectroscopy was used to monitor the reaction. The polymer tethered with GM was recovered by precipitation from diethyl ether, followed by drying in a vacuum oven at 23° C. The scheme for synthesis of the GM-tethered PAA is also described in Scheme 1b.

### Example

Synthesis of Methacryloyl beta-alanine (MBA). To a reactor containing beta-alanine (BA) and NaOH (NaOH/BA=2:1, by mole) aqueous solution, methacryloyl chloride equivalent to BA (by mole) was added at 5° C. After completion of the reaction, the solution was acidified to pH=2 with HCl (37%) and extracted three times with ethyl acetate. The extract was dried with anhydrous MgSO₄ and concentrated using a rotary evaporator to obtain white crystals. The chemical structure of MBA is shown in Scheme 1c. Additional synthetic details are described by Xie D, Faddah M, Park J G. “Novel amino acid modified zinc polycarboxylates for improved dental cements” Dent Mater 2005; 21:739-748.

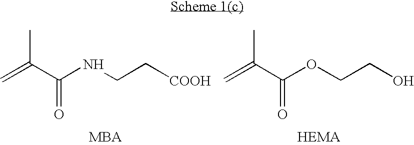

### Comparative Example

Synthesis of the linear PAA via conventional free-radical polymerization. To a flask containing AIBN and THF, a mixture of AA and THF was added dropwise. Under a nitrogen blanket, the reaction was initiated and run at 62° C. for 10 h. After the reaction was completed, the PAA was purified by precipitation using ether and drying in a vacuum oven. Additional synthetic details are described by Xie D, Faddah M, Park J G. “Novel amino acid modified zinc polycarboxylates for improved dental cements” Dent Mater 2005; 21:739-748.

### Example

Characterization of the initiator and polymers. The synthesized 4-arm initiator was characterized by melting point identification, Fourier transform-infrared (FT-IR) spectroscopy and nuclear magnetic resonance (NMR) spectroscopy. The 4-arm polymers were characterized by FT-IR, NMR and vapor pressure osmometry. Both IEM-tethered and GM-tethered polymers were identified by FT-IR and NMR spectroscopy. The melting point was measured using a digital melting point apparatus (Electrothermal IA9000 Series, Electrothermal Engineering Ltd., Essex, United Kingdom). FT-IR spectra were obtained on a FT-IR spectrometer (Mattson Research Series FT/IR 1000, Madison, Wis.). ¹H NMR spectra were obtained on an ARX-300 NMR Spectrometer using deuterated methyl sulfoxide (DMSO) as a solvent. The number average molecular weight (Mₙ) was determined using a vapor pressure osmometer (K-7000, ICON Scientific, Inc., North Potomac, Md.). The viscosity of the liquid formulated with the polymer and distilled water (50:50, by weight) was determined at 25 and 40° C. using a programmable cone/plate viscometer (RVDV-II+CP, Brookfield Eng. Lab. Inc., Middleboro, Mass.).

### Example

Formulation and preparation of specimens for strength tests.

(A) Self-cured specimens. A two-component system (liquid and powder) was used to formulate the self-cured cements, as described by Kao E C, Culbertson B M, Xie D. “Preparation of glass-ionomer cement using N-acryloyl-substituted amino acid monomers: evaluation of physical properties” Dent Mater 1996; 12:44-51. The liquid was prepared by simply mixing either 4-arm PAA or linear PAA with distilled water (50:50, by weight). Fuji II glass powder was used for making cements. The powder/liquid (P/L) was 2.7/1 (by weight, as recommended by the manufacturer).

(B) Photo-cured specimens. The light-cured cements were also formulated with a two-component system (liquid and powder), as described by Xie D, Chung I-D, Wu W, Lemons J, Puckett A, Mays J. “An amino acid modified and non-HEMA containing glass-ionomer cement” Biomaterials 2004; 25(10):1825-1830. The liquid was formulated with either IEM-tethered or GM-tethered polymer, water, 0.7% CQ (photo-initiator, by weight), 1.4% DC (activator) and 0.05% HQ (stabilizer). Fuji II LC glass powder was used to formulate the cements with a powder/liquid (P/L) ratio of 2.7. Fuji II LC kit with a P/L ratio of 3.2 (recommended by manufacturer) was used as control.

Specimens were fabricated at room temperature according to these published protocols. Briefly, the cylindrical specimens were prepared in glass tubing with dimensions of 4 mm diameter by 8 mm length for compressive strength (CS) and 4 mm diameter by 2 mm length for diametral tensile strength (DTS) tests. A split Teflon mold with dimensions of 3 mm in width×3 mm in thickness×25 mm in length was used to make rectangular specimens for flexural strength (FS) test. A transparent plastic window was used on top of the split mold for light exposure. Specimens were removed from the mold after 15 min in 100% humidity, and conditioned in distilled water at 37° C. for 24 h. Light-cured specimens were exposed to blue light (EXAKT 520 Blue Light Polymerization Unit, 9W/71, GmbH, Germany) for 1 min before conditioned in 100% humidity.

### Example

Strength measurements. Testing of specimens was performed on a screw-driven mechanical tester (QTest QT/10, MTS Systems Corp., Eden Prairie, Minn.), with a crosshead speed of 1 mm/min for CS, DTS and FS measurements. The FS test was performed in three-point bending, with a span of 20 mm between supports. The sample sizes were n=6-8 for each test.

CS was calculated using an equation of CS═P/πr², where P=the load at fracture and r=the radius of the cylinder, and DTS was determined from the relationship DTS=2P/πdt, where P=the load at fracture, d=the diameter of the cylinder and t=the thickness of the cylinder. FS was obtained using the expression FS=3 Pl/2bd², where P=the load at fracture, l=the distance between the two supports, b=the breadth of the specimen, and d=the depth of the specimen.

Statistical analysis. One-way analysis of variance (ANOVA) with the post hoc Tukey-Kramer multiple range test was used to determine significant differences of strengths among the materials in each group. A level of α=0.05 was used for statistical significance.

### Example

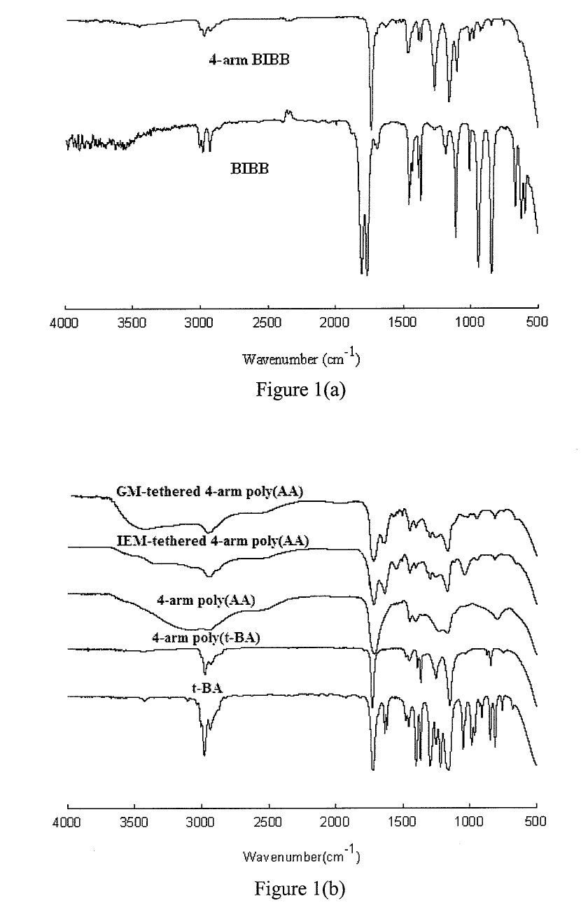

Characterization of the synthesized initiator and polymers. The purified 4-arm BIBB initiator was white crystal (yield=45%) with melting point of 135-136° C. FIG. 1a shows the spectra for both BIBB and 4-arm BIBB. The characteristic peaks are listed below: (1) BIBB (cm⁻¹): carbonyl: 1808 and 1767 (C═O stretching, strong) and 944 (C═O bending); C—Br: 848, 626 and 599 (C—Br bending); CH₃: 1459, 1371 and 1112 (CH₃ bending) and 2975-2950 (weak C—H stretching). (2) 4-arm BIBB: carbonyl: 1738 (C═O stretching, strong) and 1271 (C—O—C stretching); C—Br: 1164 (C—Br bending); CH₃: 1390, 1372, 1106 and 984 (CH₃ bending) and 2976-2933 (C—H stretching). The significant shift of carbonyl group from two peaks at 1808 and 1767 to one peak at 1738 and disappearances of 944 and 848 strongly confirmed the formation of the 4-arm BIBB.

FIG. 1b shows the FT-IR spectra for t-BA, 4-arm poly(t-BA), 4-arm PAA, IEM-tethered 4-arm PAA and GM-tethered 4-arm PAA. The t-BA shows multiple peaks in its spectrum. Among them, 1722 and 1636 are two most characteristic peaks associated with carbonyl and carbon-carbon double bond, respectively. In contrast, disappearance of the peak at 1636 cm⁻¹ in the spectrum for the 4-arm poly(t-BA) confirmed the completion of polymerization. After hydrolysis of the 4-arm poly(t-BA), a broad and significant peak at 3600-2300 cm⁻¹ and a strong but wider peak at 1714 cm⁻¹ can be observed as compared to poly(t-BA). The former is the typical peak for hydroxyl group on carboxylic acids (OH stretching) whereas the latter is the characteristic peak for carbonyl stretching on PAA. In contrast, the IEM-tethered 4-arm PAA shows four typical peaks: 3600-2400 cm⁻¹ (OH stretching on COOH); 1717 (carbonyl, C═O stretching on COO and CONH, where both carbonyl peaks were overlapped); 1636 (C═C bending); and 1553 (amide II, CONH). For the GM-tethered PAA, four characteristic peaks are: 3600-2400 cm⁻¹ (OH stretching on COOH); 3434 (OH on tethered methacrylate); 1716 (C═O stretching on COO); and 1636 (C═C bending). It is apparent that the peak at 3434 cm⁻¹ on the GM-tethered 4-arm PAA and the peak at 1553 cm⁻¹ on the IEM-tethered 4-arm polymer identified the difference between these two polymers. The peak at 1636 cm⁻¹ identified the difference between the 4-arm PAA and IEM/GM-tethered 4-arm PAA.

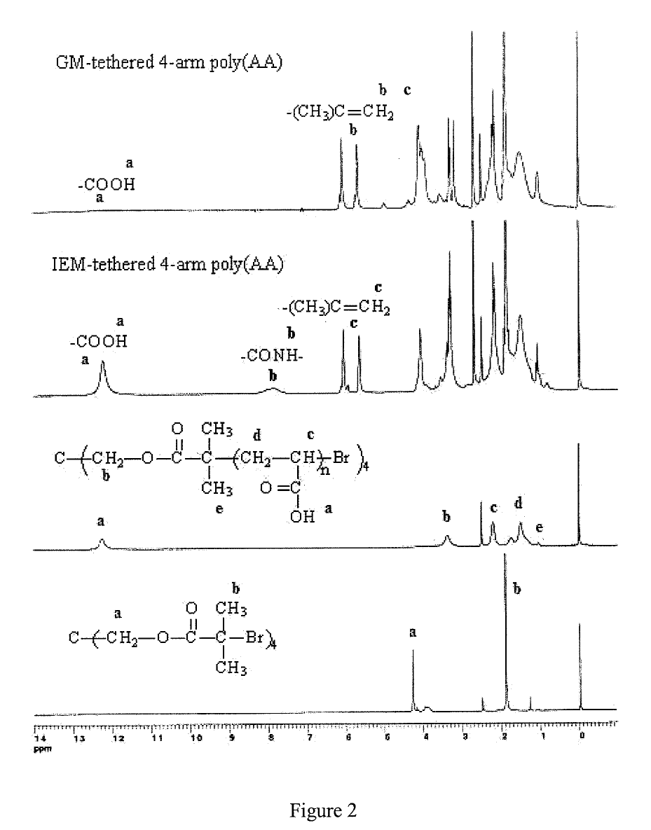

FIG. 2 shows the ¹H NMR spectra for the 4-arm BIBB, 4-arm PAA, IEM-tethered 4-arm PAA and GM-tethered 4-arm PAA. The chemical shifts of the 4-arm BIBB initiator were found as follows (ppm): a: 4.3 (CH₂) and b: 1.9 (CH₃). The chemical shifts of the 4-arm PAA were listed below (ppm): a: 12.25 (COOH); b: 3.4 (CH₂); c: 2.25 (CH); d: 1.8 and 1.55 (CH₂); and e: 1.1 (CH₃). The single peak at 2.50 (between b and c) was the chemical shift for solvent DMSO. All the spectra contain this peak. The typical chemical shifts for the IEM-tethered 4-arm PAA were shown below (ppm): a: 12.25 (COOH), b: 7.9 (CONH), and c: 6.15 and 5.75 (C═CH₂). The characteristic chemical shifts at 7.9, and 5.75 and 6.15 identified the difference between 4-arm PAA and IEM-tethered 4-arm PAA. The typical chemical shifts for the GM-tethered 4-arm PAA were: a: 12.30 (COOH), b: 5.70 and 6.10 (C═CH₂), and c: 3.25 (OH). The chemical shift for COOH on GM-tethered 4-arm PAA was weak but broad. The characteristic chemical shifts at 3.25, 5.70 and 6.10 identified the difference between the 4-arm PAA and GM-tethered 4-arm PAA.

### Example

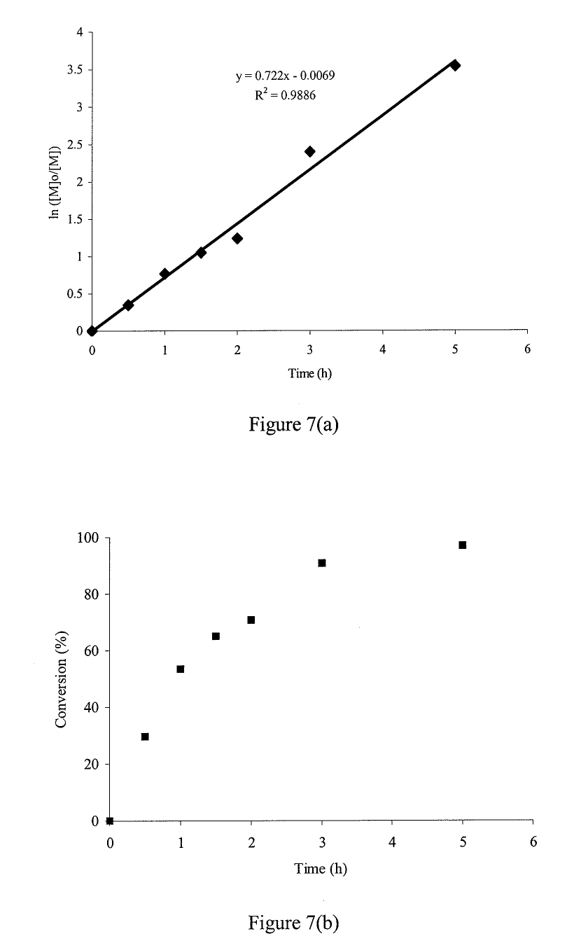

Synthesis of the 4-arm PAA. Atom-transfer radical polymerization (ATRP), a recently developed technology for controlled radical polymerization, is capable of making various architectures such as star polymers and block copolymers. Additional synthetic details are described by K. Matj aszewski and J. Xia, “Atom transfer radical polymerization” Chem. Rev. 101 (2001) 2921-2990. FIG. 7 shows a semi-logarithmic plot of the ATRP of t-BA in dioxane (a) and a kinetic plot of monomer to polymer conversion versus time (b). The polymerization was initiated by the 4-arm BIBB, catalyzed by CuBr-PMDETA complex and run at 120° C. The plot of ln([M]₀/[M]) versus time (FIG. 7(a)), where [M]₀=the initial concentration of the monomer and [M]=the monomer concentration at any time, is almost linear, suggesting that the polymerization propagation was constant throughout the reaction or in other words, a constant concentration of growing radicals reflects a first-order kinetics. From the kinetic plot of monomer to polymer conversion versus time (FIG. 7(b)), it appears that the monomer conversion increased with time. The reaction in dioxane took 3 h to reach a 90% conversion and 5 h to reach a 97% conversion.

In order to demonstrate that the t-BA was polymerized only by ATRP but not by heat-initiated conventional free-radical polymerization, a parallel experiment without any initiator involved was conducted under the same condition. It was found that no polymer was generated within 8 h, which suggest that the poly(t-BA) was polymerized by the ATRP reaction. The 4-arm PAA was prepared by hydrolysis of the poly(t-BA) in a mixed solvent of dioxane and aqueous HCl (37%) for 8-12 h under refluxed condition, followed by dialysis against water until the pH reached neutral. Additional synthetic details are described by L. Stanislawski, X. Daniau, A. Lauti A, and M. Goldberg, “Factors responsible for pulp cell cytotoxicity induced by resin-modified glass ionomer cements” J. Biomed. Mater. Res. 48(3) (1999) 277-88.

The molecular weights (MWs) of the synthesized 4-arm PAA via ATRP and linear PAA via conventional free-radical polymerization were characterized using VPO and shown in Table 1. Table 1 shows the MW, conversion and viscosity of the three 4-arm PAAs and one linear PAA. The MWs of the 4-arm PAAs synthesized via ATRP were 15,701, 18,066 and 21,651 Daltons whereas the MW of the linear PAA synthesized via conventional free-radical polymerization was 9,704. The conversions of the monomer to polymer were determined using FT-IR spectra and they wee all greater than 97%. The viscosities were measured using a cone & plate viscometer and shown in Table 1. At 25° C., the viscosities of the two 4-arm PAAs (MW=15,701 and 18,066) were 48.2 and 148.6 (cp) but the solutions of the other 4-arm PAA (MW 21,651) and the linear PAA (MW=9,704) were too viscous to be measured for their viscosities. At 40° C., the viscosities of both 4-arm PAA (MW=21,651) and linear PAA could be measured but they were much higher than the other two 4-arm PAAs. It is observed that the 4-arm PAA even with a MW of 18,066 showed a lower viscosity value than the linear PAA (not measurable), although the latter's MW was only 9,704. It appears that increasing MW increased the viscosity of polymer aqueous solution, and that the star-shape structure of the 4-arm PAA may contribute to a lower viscosity as compared to the linear PAA, even though the linear polymer had a lower MW. These results suggest that the more spherical nature of the multifunctional core molecule in the 4-arm PAA improves viscosity even at high molecular weight.

TABLE 1
Conversion, MW and viscosity of synthesized polymers.

| | | | | Vis- | Vis- |
|---|---|---|---|---|---|
| | | Conversion ¹ | MW ² | cosity ³ | cosity ⁴ |
| Example | Polymer | (%) | (Dalton) | (cp) | (cp) |
| A | 4-arm PAA | 99.4 | 15,701 | 49.2 | 11.2 |
| B | 4-arm PAA | 97.5 | 18,066 | 148.6 | 67.3 |
| C | 4-arm PAA | 97.0 | 21,651 | NM ⁵ | 980 |
| D | Linear PAA ⁶ | 99.9 | 9,704 | NM ⁵ | 1890 |

¹ Conversion (%) was measured from FT-IR spectra;
² MW (number average) was determined in DMF via a vapor pressure osmometer;
³ Viscosity of the aqueous polymer solution (PAA:distilled water = 1:1, by weight) was measured using a cone &plate viscometer at 25° C.
⁴ Viscosity was measured using a cone &plate viscometer at 40° C.
⁵ NM stands for the viscosity that was not measurable at the given temperature due to gel formation. Specimens were conditioned in distilled water at 37° C. for 24 h;
⁶ Linear PAA was synthesized via conventional free-radical polymerization using 1% AIBN as initiator.

### Example

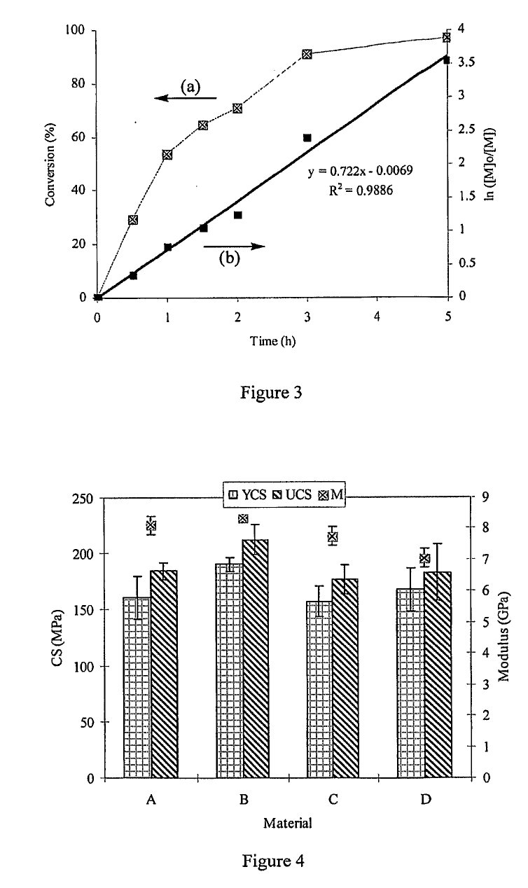

Synthesis and hydrolysis of the 4-arm poly(t-BA). It is known that almost all the poly(carboxylic acid)s being used in current dental GICs are linear polymers and synthesized via conventional free radical polymerization. So far no reports have been found on studies of different architectures of the polyacids for GIC applications. One of the main reasons may be attributed to the fact that it is impossible to synthesize the polymers with different architectures by using conventional free-radical polymerization techniques. Atom-transfer radical polymerization (ATRP), a recently developed technology for controlled radical polymerization, is capable of making various architectures such as star polymers and block copolymers. By using such a technique, we were able to synthesize novel star-shape PAA in this study. FIG. 3 shows a semi-logarithmic plot of the ATRP of t-BA in dioxane and a kinetic plot of monomer to polymer conversion versus time. The polymerization was initiated by the 4-arm BIBB, catalyzed by CuBr-PMDETA complex and run at 120° C. The plot of ln ([M]₀/[M]) versus time, where [M]₀=the initial concentration of the monomer and [M]=the monomer concentration at any time, is almost linear, suggesting that the polymerization propagation is constant throughout the reaction or in other words, a constant concentration of growing radicals reflects a first-order kinetics. From the kinetic plot of monomer to polymer conversion versus time, it is apparent that the monomer conversion increases with time. The reaction in dioxane took 3 h to reach a 90% conversion and 5 h to reach a 97% conversion. In order to make sure that the t-BA was polymerized only by ATRP but not by heat-initiated conventional free-radical polymerization, a parallel experiment without any initiator involved was conducted under the same condition. It was found that no polymer was generated within 8 h, which indicates that the poly(t-BA) was only polymerized by the ATRP reaction.

The 4-arm PAA was prepared by hydrolysis of the poly(t-BA) in a mixed solvent of dioxane and aqueous HCl (37%) for 8-18 h under refluxed condition, followed by dialysis against water until the pH reached neutral. We noticed that the higher the MW of the 4-arm poly(t-BA) the longer the time was needed for hydrolysis. The duration depends upon the MW of the polymer. In the case of the poly(t-BA) with MW of 15,701, it took about eight hours to complete the hydrolysis. For the poly(t-BA) with MW of 21,651, however, eighteen hours were required for completing the hydrolysis, which is probably due to the bulky long chains from the 4-arm poly(t-BA).

### Example

Synthesis of the IEM-tethered and GM-tethered 4-arm PAAs. The reaction between IEM and carboxylic acid on PAA was quite efficient. The reaction took only two hours to complete. Disappearance of the isocyanate group at 2250 cm⁻¹ by FT-IR monitoring confirmed the completion of the reaction. The reaction between GM and carboxylic acid on PAA took about fourteen hours to complete. Disappearance of the epoxy group on GM at 761 cm⁻¹ confirmed the completion of the tethering reaction. The completion of the tethering for both reactions was also confirmed by the fact that yields were greater than 95%.

### Example

Selection of the 4-arm PAA for methacrylate tethering. In this study, we synthesized three 4-arm PAA polymers A, B and C with MW of 15,701, 18,066 and 21,651, respectively. The viscosities of these polymers in water (50/50, by weight) were also determined at 25° C. and 40° C., respectively. The values (cp) at 25° C. were in the order of C (too high, not measurable)>B (148.6)>A (49.2), corresponding to their decreased MW. The viscosities at 40° C. (elevated temperature) showed that C was much higher than both B and A. The compressive strengths (CS) of the corresponding cements formulated with Fuji II glass fillers are shown in FIG. 4. The cement B with MW of 18,066 showed the highest yield CS (YCS, 190.0 MPa), ultimate CS (UCS, 212.2 MPa) and modulus (M, 8.33 GPa), followed by the A (160.9, 184.1 and 8.11) and the C (157.1, 176.9 and 7.74). Due to its suitable viscosity and highest CS, the polymer B was selected for methacrylate tethering. For comparison, the CS of the linear PAA-composed cement D (liquid viscosity at 25° C.=not measurable and at 40° C.=1350 cp) was also determined. The CS values for D were 167 MPa in YCS, 183 MPa in UCS and 7.04 GPa in M. It is worthy to point out that it was very difficult to make the specimens from both C and D because of their high solution viscosities. Strong hydrogen bonds are probably attributed to the higher viscosities of both C and D.

### Example

Tethering of IEM or GM onto the 4-arm PAA for light-curable GICs. IEM tethering for pendant methacrylate functionalities on poly(carboxylic acid)s has been applied in our previous research and it was very successful, because the reaction was fast and clean and the yield was high. However, the disadvantages for using this isocyanate-containing methacrylate are its high cost and toxicity. The solubility of the IEM-tethered polyacid in water is low as well. To overcome the low solubility of the IEM-tethered PAA in water, amphiphilic comonomers such as HEMA or amino acid derivatives, as described by Xie D, Chung I-D, Wu W, Lemons J, Puckett A, Mays J. “An amino acid modified and non-HEMA containing glass-ionomer cement” Biomaterials 2004; 25(10):1825-1830; Xie D, Faddah M, Park J G. “Novel amino acid modified zinc polycarboxylates for improved dental cements” Dent Mater 2005; 21:739-748; have been incorporated. In this study, both HEMA and MBA were used as a comonomer for comparison. Regarding GM tethering, no reports have been found so far on using this reagent for GIC modifications. By looking at the tethered chemical structure (see FIG. 1), each GM molecule produces one extra hydroxyl group when epoxy group on GM reacts with carboxyl group on PAA. Unlike IEM-tethering, these hydroxyl groups should make the GM-tethered PAA less hydrophobic or to say the least they should not change the original hydrophilicity of the PAA much.

Table 2 shows the effects of different comonomer and grafting agent on compressive properties. Codes E, F, G and H stand for the cements tethered with 35%, 35%, 50%, and 50% GM and mixed with HEMA, MBA, HEMA and MBA, respectively. By comparing E and F, the MBA, acid-containing comonomer, exhibited significantly high YCS, M and UCS. It appears that both YCS and M increased even more significantly, which can be attributed to salt-bridges formations contributed by MBA, because salt-bridges often make the cements more brittle and it is known that brittle materials are high in yield strength and modulus. That is why the MBA-containing cement was higher in YCS and M than the HEMA-containing cement. The same principle is applied to G and H. By comparing E and G or F and H, a higher grafting ratio gave higher UCS but not necessarily YCS and M, which can be explained as the reason that a higher grafting ratio means more resin components incorporated and thus contributes to lower YCS and modulus.

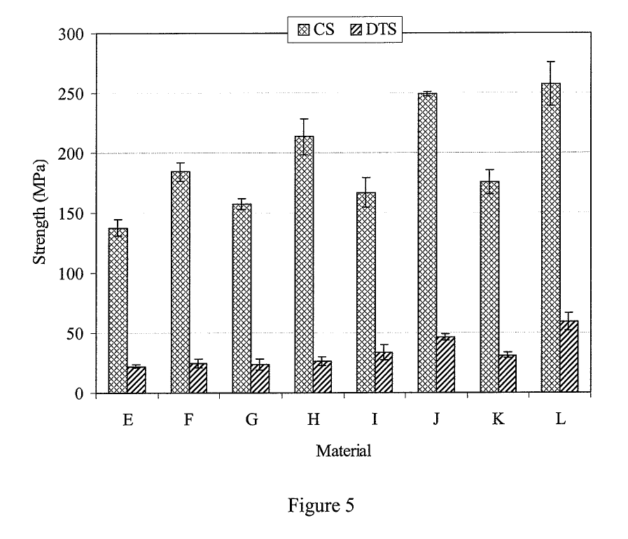

In the case of IEM-tethering I J, K and L, the trend was pretty similar to that for the GM-tethered cements. As shown in Table 2, J was much higher in YCS, modulus and UCS than I whereas L was much higher than K. For the HEMA-containing cements, the 50% IEM-tethered cement (K) was statistically the same in YCS and UCS as the 35% IEM-tethered cement (I) but was lower in M. The similar result was found to the MBA-containing cements (L and J). However, by comparing the GM- and IEG-tethered cements, it is apparent that all the IEM-tethered cements were higher in YCS, modulus and UCS than corresponding GM-tethered cements. For example, the 50% IEM-tethered cement with MBA (175.1 MPa in YCS, 6.5 GPa in modulus and 257 MPa in UCS) was 22%, 20% and 21% higher than corresponding the 50% GM-tethered cement with MBA (144.1, 5.4 and 213.2). This obvious difference can be attributed to nature difference between IEM and GM-tethered cements, because the former contained more hydrophobic IEM-tethered 4-arm PAA whereas the latter contained more hydrophilic GM-tethered 4-arm PAA due to the extra hydroxyl groups. These hydroxyl groups can keep more water around, which make the cements relative weaker in strength because the cement somehow behaves like a hydrogel material. As we know, polymeric hydrogel materials often show lower mechanical strengths due to their hydrophilic nature, as described by Ratner B D, Hoffman A S, Schoen F J, Lemons J E. Biomaterials Science, An Introduction to Materials in Medicine, San Diego, Calif.: Academic Press; 1996. FIG. 5 shows both UCS and DTS values of the cements discussed above. Not only CS but also DTS showed the same trends in mechanical strengths to these cements. The order of DTS (MPa) was: L (58.9±7.2)>J (46.7±2.7)>I(33.9±6.4)>K(31.3±1.9)>H(26.5±3.6)>F(24.7±3.8)>G(23.5±4.4)>E (22.1±1.2). Both CS (257.1±18 MPa) and DTS (58.9±7.2 MPa) of the 50% IEM-tethered cement with MBA as comonomer was the highest among all the cements.

TABLE 2
Effects of comonomer and tethering type on compressive properties

| | | Graft | Grafting | | | | | | |
|---|---|---|---|---|---|---|---|---|---|
| Example | Comonomer | Type | Ratio | YCS [MPa]¹ | | Modulus [GPa] | | UCS [MPa]² | |
| E | HEMA | GM | 35% | 54.2 | (2.1)^{3,a} | 2.29 | (0.18)^{e} | 137.3 | (6.8)^{g} |
| F | MBA | GM | 35% | 134.9 | (6.6)^{b} | 6.10 | (0.21)^{f} | 184.1 | (7.9)^{h} |
| G | HEMA | GM | 50% | 53.0 | (3.7)^{a} | 2.65 | (0.25)^{e} | 157.4 | (4.4)^{g,i} |
| H | MBA | GM | 50% | 144.1 | (8.2)^{b} | 5.40 | (0.37) | 213.2 | (15) |
| I | HEMA | IEM | 35% | 68.1 | (4.2)^{c} | 3.12 | (0.32) | 166.7 | (12)^{i,j} |
| J | MBA | IEM | 35% | 173.5 | (1.1)^{d} | 7.10 | (0.07) | 249.5 | (1.8)^{k} |
| K | HEMA | IEM | 50% | 66.8 | (7.5)^{a,c} | 2.63 | (0.20)^{e} | 175.2 | (10)^{h,j} |
| L | MBA | IEM | 50% | 175.1 | (4.9)^{d} | 6.50 | (0.54)^{f} | 257.1 | (18)^{k} |

¹YCS = CS at yield;
²UCS = ultimate CS;
³Entries are mean values with standard deviations in parentheses and the mean values with the same superscript letter were not significantly different (p > 0.05). Specimens were conditioned in distilled water at 37° C. for 24 h.

### Example

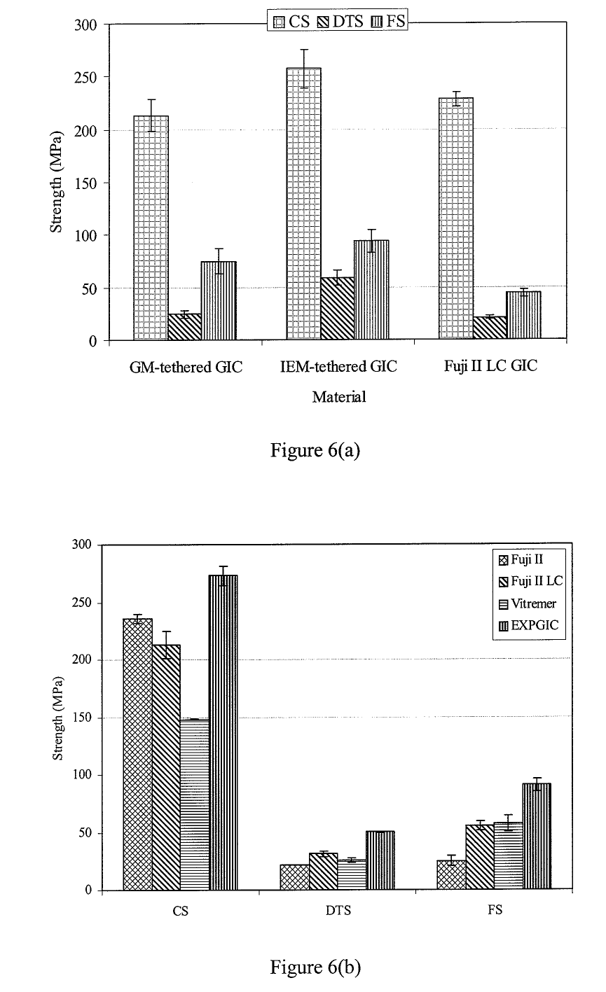

Mechanical strength comparison among the cements described herein and commercial Fuji II LC. The CS, DTS and FS of illustrative Examples were compared with those of commercial Fuji II LC cement. The results in FIG. 6a show that the IEM-tethered cement exhibited significantly higher FS, DTS, and CS than Fuji II LC. The GM-tethered cement exhibited significantly higher FS and statistically similar DTS and CS compared to Fuji II LC. In addition, FIG. 6b shows the CS, DTS and FS values for Example M (GM-tethered 4-arm PAA) compared to commercial Fuji II, Fuji II LC, and Vitremer cements. The observed strengths (MPa) for the cements are shown in Table 3. Example M refers to a cement with a P/L ratio of 2.7/1, GM-grafting ratio=50% and PIW=75/25 (see, Xie, D., Yang, Y., Zhao, J., Park, J-G., Zhang, J.-T. “A Novel Comonomer-Free Light Cured Glass-lonomer Cement for Reduced Cytotoxicity and Enhanced Mechanical Strengths” Dental Materials in press.), the disclosure of which is incorporated herein by reference in its entirety

TABLE 3
FS, DTS, and CS for Examples compared to commercial cements.

| Example | FS (MPa) | DTS (MPa) | CS (MPa) |
|---|---|---|---|
| IBM-tethered | 93.9 ± 11 | 58.9 ± 7.2 | 257.1 ± 18 |
| GM-tethered | 74.7 ± 12 | 24.4 ± 3.6 | 213.3 ± 15 |
| Example M | 90.8 ± 5.5 | 50.2 ± 0.5 | 272.9 ± 8.4 |
| Fuji II | 25.1 ± 4.8 | 21.6 ± 0.1 ^{(b)} | 235.6 ± 4.4 ^{(a)} |
| Fuji II LC | 55.8 ± 4.1 ^{(c)} | 31.2 ± 2.2 | 212.7 ± 12 ^{(a)} |
| Vitremer | 57.8 ± 6.9 ^{(c)} | 25.6 ± 0.6 ^{(b)} | 148 ± 0.6 |

^{(a)} not significantly different (p > 0.05);
^{(b)} not significantly different (p > 0.05);
^{(c)} not significantly different (p > 0.05).

The light-curable 4-arm star-shape PAA was synthesized via ATRP and showed a lower viscosity as compared to the corresponding linear counterpart that was synthesized via conventional free-radical polymerization. Without being bound by theory, it is suggested that the spherical nature of the 4-arm star-shape PAA may account for the difference in observed viscosity. Both GM-tethered and IEM-tethered variants of the 4-arm PAA-constructed LCGICs showed significantly high mechanical strengths than conventional cements. It was also observed that the MBA-containing cement variants exhibited much higher CS than the HEMA-containing cement variants. Without being bound by theory, it is also suggested that a salt-bridge contribution of the MBA may account for the improved CS. The IEM-tethered cement variants showed much higher mechanical strengths than the GM-tethered cement variants. Without being bound by theory, it is also suggested that a hydrophobicity difference between the two corresponding polymers may account for the improved mechanical strengths. The selected cements described herein showed 13% improvement in CS, 178% improvement in DTS, and/or 123% improvement in FS over the conventional cement prepared from FUJI II LC.

The results in Table 4 show that the polyfunctional core molecules and prepolymer compounds described herein, including poly(acrylic acid) tethered with pendent methacrylate to formulate the LCGIC improves the mechanical strengths and wear resistance of the GICs. The 4-arm star poly(acrylic acid) Example was improved by 48% in CS, 76% in DTS, 95% in FS and 60% in FT higher than Fuji II LC cement. The Example also showed higher wear-resistance (97.5 μm³ cycle⁻¹) than Fuji II LC (11525 μm³ cycle⁻¹). Although the Example was 5% lower in CS, 20% higher in DTS, 20% lower in FS and 15% lower in FT than Filtek P60 posterior composite resin, it showed surprisingly improved (97.5 μm³ cycle⁻¹) wear-resistance than Filtek P60 (545 μm³ cycle⁻¹). These results indicate that it is feasible to make glass-ionomer cements to become a restorative with wear-resistance and mechanical strengths comparable to current posterior composite resins.

TABLE 4
CS, DTS, FS, FT and wear of 4-arm, Fuji II LC and FiltekP60

| | CS | | DTS | FS | | FT | Wear | |
|---|---|---|---|---|---|---|---|---|
| Example¹ | [MPa] | | [MPa] | [MPa] | | [MPa · m^{-1/2}] | (volume loss) | |
| 4-arm | 323.3 | (11) | 61.7 (5.3) | 103.5 | (0.7) | 1.45 (0.05) | 0.039 | (0.01) |
| Fuji II LC | 219.1 | (1.7) | 34.9 (2.9) | 53.0 | (2.8) | 0.91 (0.03) | 4.61 | (0.44) |
| P-60 | 349.1 | (18) | 43.9 (4.2) | 157.6 | (2.6) | 1.71 (0.07) | 0.218 | (0.05) |

¹4-arm: The 4-arm star-shape poly(acrylic acid)-composed LCGIC, where Filler = Fuji II LC filler, Grafting ratio = 50%, P/W ratio = 75/25, and P/L ratio = 2.7; Fuji II LC: Fuji II LC LCGIC, where P/L ratio = 3.2; P-6Q: Filtek P60 posterior composite resin; All the specimens were light cured for 1-2 min. The 4-arm and Fuji II LC GICs for CS, DTS, FS, and FT tests were conditioned in distilled water at 37° C. for 1 week prior to testing. The 4-arm and Fuji II LC for wear-resistance were tested on a three-body machine after 24 h storage in water at 37° C. All the cured specimens for P-60 were tested after 1 h under dry conditions. The wear cycle = 400,000.

### Example

Synthesis of the GM-tethered 4-arm PAA. The reaction between GM and carboxylic acid on PAA took about fourteen hours to complete. Disappearance of the epoxy group on GM at 761 cm⁻¹ (FT-IR) confirmed the completion of the tethering reaction. The completion of the tethering of GM was also confirmed by the fact that the yield was greater than 95%.

### Method Example

Significance of tethering of GM onto the 4-arm PAA. It is believed that the main difference between RMGICs and CGICs is their liquid composition as described by A. D. Wilson, “Resin-modified glass-ionomer cement” Int. J. Prosthodont. 3 (1990) 425-429. The liquid in RMGICs is composed of HEMA, photo-initiators, water, and a poly(alkenoic acid) having pendent in situ polymerizable methacrylate on its backbone or a mixture of poly(alkenoic acid) and methacrylate-containing monomer/oligomer. The liquid in CGICs consists of only hydrophilic poly(alkenoic acid) and water. Due to introduction of hydrophobic methacrylate functionality, amphiphilic monomers such as HEMA have to be incorporated into the RMGIC liquid formulation to enhance the solubility of the hydrophobic poly(alkenoic acid) in water. Without these amphiphilic small molecules like HEMA, it is difficult if not impossible to formulate RMGICs by using current technologies. It has shown that tethering GM onto the poly(alkenoic acid) backbone can increase water-solubility of the polyacid because of introduction of hydroxyl groups as compared to 2-isocyanatoethyl methacrylate (IEM)-tethered poly(alkenoic acid), as described by as described by D. Xie, J. G. Park, and M. Faddah, J. Biomater. Sci. Polym. Edn. in press; S. B. Mitra, J. Dent. Res. 70 (1991) 72-74; D. Xie, B. M. Culbertson, and W. M. Johnston, J. M. S. Pure Appl. Chem. A35(10) (1998) 1631-1650; D. Xie, I-D. Chung, W. Wu, J. Lemons, A. Puckett, and J. Mays, “An amino acid modified and non-HEMA containing glass-ionomer cement” Biomaterials 25(10), (2004) 1825-1830. The chemical structure of the GM-tethered 4-arm PAA as shown in FIG. 1b, indicates that each GM molecule produces one extra hydroxyl group when the epoxy group on GM reacts with the carboxyl group on PAA. Unlike IEM-tethering, these hydroxyl groups may make the GM-tethered PAA less hydrophobic or at least not increase the hydrophilicity of the PAA. It is appreciated however that additional hydroxyl groups have the potential to reduce the mechanical strength and increase the viscosity due to their ability to absorb water and serve as a hydrogel. In contrast, those same hydrogen bonds make a contribution to hydrogen bond formation, thus increasing viscosity.

### Method Example

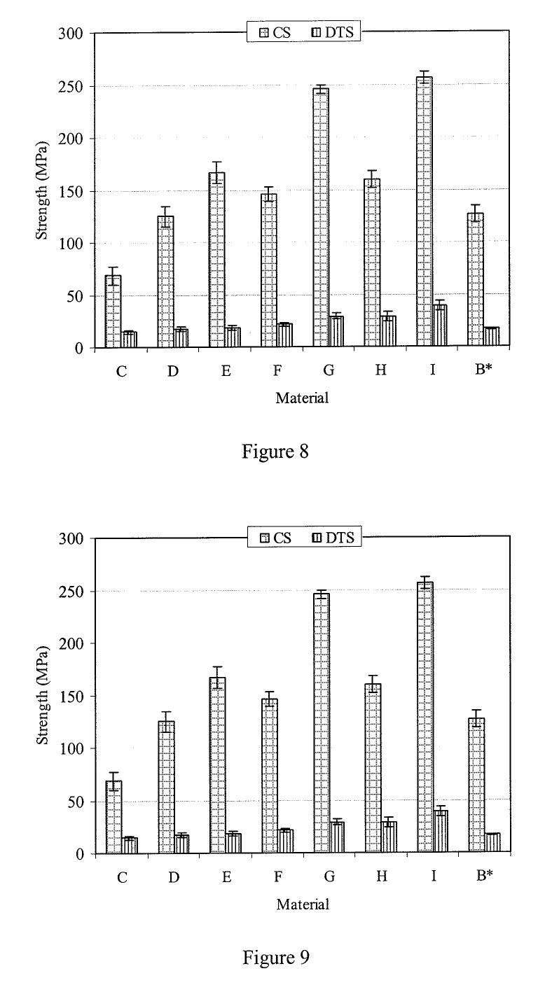

Effects of polymer/water ratio and grafting ratio on compressive properties. To study the effects of P/W ratio (by weight) and grafting ratio (by mole) on strengths, seven liquid solutions (C to I) based on the 4-arm PAA tethered with GM and one liquid solution (B*) based on the linear PAA tethered with GM were formulated. Three P/W ratios including 50/50, 60/40 and 75/25 and three grafting ratios including 35%, 50% and 70% were studied. Table 5 and FIG. 8 show the results of CS and DTS of the cements prepared from the above formulations. The cements C, D and E represent the 35% GM-tethered 4-arm PAAs with the P/W ratio at 50/50, 60/40 and 75/25. It is observed that increasing P/W ratio significantly increased yield compressive strength (YCS), modulus (M) and ultimate compressive strength (UCS), indicating that a higher polymer concentration may enhance the mechanical strength of the relatively hydrophilic GM-tethered PAA cement. The cement C showed the lowest YCS (47.5 MPa), M (2.65 GPa) and UCS (68.5 MPa), suggesting that at 50/50, the hydrophilic characteristic of the GM-tethered PAA prevails and the cement behaves like a hydrogel. However, increasing polymer content in water overcomes that property exhibited by the hydroxyl groups from the GM-tethered PAA and makes the cement stronger.

### Method Example

The effect of grafting ratio on the strength was studied by changing the grafting ratio from 35% to 70%. It was observed that at P/W=60/40 increasing grafting ratio significantly increased YCS and UCS but not necessarily M. However, at 75/25, increasing grafting ratio did significantly increase the CS values from 35% to 50% but did not significantly change the CS when the ratio reached 70%. However, there was no statistical difference between the 50% and 70% GM-tethered cements at 75/25. The highest strength values were observed as falling between the 50% and 70% GM-tethered 4-arm PAA cements at P/W ratio=75/25, a shown in Table 5. These results support the feasibility of eliminating low MW comonomers in RMGIC formulations, which may improve the biocompatibility of conventional light-cured GICs. In contrast, the linear PAA (B*) that was synthesized via conventional free-radical polymerization showed much lower strengths (YCS=105.4 MPa, M=5.43 GPa and UCS=124.5) than those for corresponding 4-arm PAA cement (G, 170.3, 6.62 and 245.8). The data from DTS showed the similar trend to those from CS. The order of DTS (MPa) was: I(39.5±4.6)>G (29.3±2.4)>H (29.1±4.5)>F (21.3±2.0)>E (18.4±2.2)>D (17.3±2.2)>N (14.4±2.0). Both CS (256.0 MPa) and DTS (39.5 MPa) of the 70% GM-tethered cement at a P/W ratio of 75/25 were the highest among all the GM-tethered 4-arm PAA-constructed cements.

TABLE 5
Effects of polymer/water ratio and GM grafting ratio on compressive properties

| | P/W | Grafting | | | Modulus | | | | |
|---|---|---|---|---|---|---|---|---|---|
| Example | Ratio | Ratio | YCS [MPa]¹ | | [GPa] | | UCS [MPa]² | | Viscosity³ |
| C | 50/50 | 35% | 47.5 | (8.2)³ | 2.65 | (0.82) | 68.5 | (7.2) | 75.6 |
| D | 60/40 | 35% | 81.8 | (6.0) | 5.00 | (0.25)^{b,c} | 124.8 | (9.4)^{e} | 275.2 |
| E | 75/25 | 35% | 143.2 | (2.7) | 6.43 | (0.18)^{d} | 166.8 | (9.9)^{f} | 3323 |
| F | 60/40 | 50% | 91.9 | (4.2) | 4.85 | (0.18)^{b} | 146.5 | (6.9) | 171.5 |
| G | 75/25 | 50% | 202.3 | (7.2) | 6.84 | (0.45)^{d} | 272.9 | (8.5)^{g} | 1764 |
| H | 60/40 | 70% | 105.5 | (7.9)^{a} | 5.19 | (0.25)^{c} | 159.7 | (7.6)^{f} | 206.4 |
| I | 75/25 | 70% | 197.2 | (11) | 6.67 | (0.18)^{d} | 286.8 | (12)^{g} | 2094 |
| B*⁴ | 75/25 | 50% | 105.4 | (7.7)^{a} | 5.43 | (0.34)^{c} | 126.5 | (7.7)^{e} | 6830 |

¹YCS = CS at yield;
²UCS = ultimate CS;
³Entries are mean values with standard deviations in parentheses and the mean values with the same superscript letter were not significantly different (p > 0.05).
⁴B* = linear PAA, which was synthesized via conventional free-radical polymerization and tethered with GM. Specimens were conditioned in distilled water at 37° C. for 24 h.

### Method Example

Effect of glass powder/polymer liquid ratio on compressive properties. The glass powder/polymer liquid (P/L) ratio is an important parameter in formulating GICs. A higher P/L ratio may result in higher mechanical strengths, especially CS, but it may also shorten working time. It is appreciated that working time is less of an issue for a light-curable GIC system, and therefore a higher P/L ratio may be used in LCGICs, such as the filler FUJI II LC (3.2). The effect of three P/L ratios (2.2, 2.7 and 3.0) on CS is shown in Table 6. A significant increase in YCS, M and UCS was observed when the P/L ratio was increased from 2.2 to 2.7 but not from 2.7 to 3.0. No statistical difference in YCS, M and UCS was found between 2.7 and 3.0. A formulation with a P/L ratio of 2.7/1 for the Examples was used to make experimental cements derived from GM-tethered 4-arm PAA.

TABLE 6
Effects of P/L ratio and aging on compressive properties

| | | | |
|---|---|---|---|
| Parameter | YCS [MPa]¹ | UCS [MPa]² | Modulus [GPa] |

Effect of P/L ratio⁴

| | | | | | | |
|---|---|---|---|---|---|---|
| 2.2 | 144.2 | (1.3)³ | 204.7 | (1.8) | 5.86 | (0.30) |
| 2.7 | 202.3 | (7.2) | 272.9 | (8.5) | 6.89 | (0.45)^{c} |
| 2.7 | 164.0 | (1.1)^{a} | 256.0 | (5.8)^{b} | 6.89 | (0.33)^{c} |
| 3.0 | 179.4 | (1.9) | 244.2 | (2.1) | 6.94 | (0.21)^{c} |
| 3.0 | 170.4 | (2.1)^{a} | 244.2 | (2.1)^{b} | 6.94 | (0.21)^{c} |

Effect of aging⁵

| | | | | | | |
|---|---|---|---|---|---|---|
| 1 h | 78.1 | (2.8) | 209.2 | (6.5) | 2.59 | (0.02) |
| 1 d | 164.0 | (1.1) | 256.0 | (5.8) | 6.89 | (0.33) |
| 1 w | 252.9 | (3.1) | 329.7 | (11) | 8.12 | (0.29) |

¹YCS = CS at yield;
²UCS = ultimate CS;
³Entries are mean values with standard deviations in parentheses and the mean values with the same superscript letter were not significantly different (p > 0.05);
⁴Grafting ratio = 70% and P/W ratio = 75/25; Specimens were conditioned in distilled water at 37° C. for 24 h;
⁵Grafting ratio = 70%, P/W ratio = 75/25 and P/L ratio = 2.7. Specimens were conditioned in distilled water at 37° C. prior to testing.

### Method Example

Aging. It has been reported that GICs increase their strengths with time due to the continuing formation of salt-bridges, as described by C. L. Davidson, and I. A. Mjör, “Advances in glass-ionomer cements” (Quintessence Publ Co., Chicago, Ill., 1999). The optimal 70% GM-tethered 4-arm PAA cement was conditioned at 37° C. in distilled water for 1 h, lday and 1 week, followed by CS determinations. As shown in Table 6, the compressive strengths were significantly increased from 78.1 to 252.9 MPa in YCS, 2.59 to 8.12 GPa in M, and 209.2 to 329.7 MPa in UCS within one week.

Comparison between the experimental cement and commercial control. The FS of the optimal experimental cement was measured and compared to the measured CS, DTS and FS of commercial FUJI II LC cement. The strengths of both cements were determined after conditioning in distilled water at 37° C. for 24 h. As shown in FIG. 9, the light-cured experimental cement showed significantly higher CS (256.0±5.8 MPa), DTS (39.5±4.6 MPa) and FS (98.4±5.0 MPa) as compared to corresponding 228.2±6.4, 21.2±1.1 and 44.2±3.4 for FUJI II LC.

### Method Example

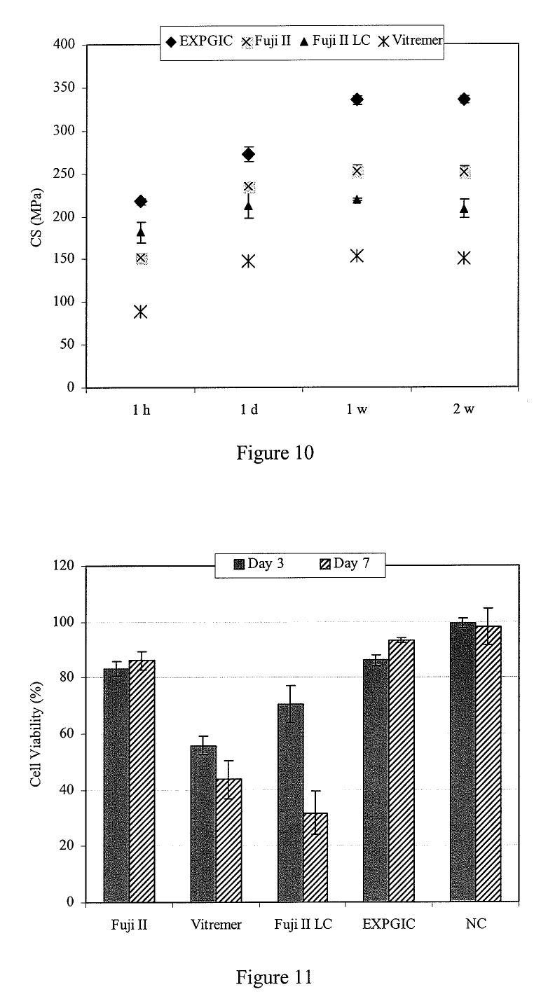

Mechanical strength comparison. The mechanical strength (CS, DTS and FS) between Example M and commercial Fuji II (conventional GIC), Fuji II LC (light-cured GIC) and Vitremer (light-cured GIC) (FIG. 10). Table 7 shows the details of strength changes of these cements in the course of aging, including yield compressive strength (YS), modulus (M), and ultimate compressive strength (UCS). Example M showed significantly higher CS, DTS and FS as compared to the tested commercial cements as shown in Table 6. Higher mechanical strengths is exhibited by Example M. Without being bound by theory, it is suggested that because Example M has a comonomer-free and pendent hydroxyl group-containing system, the polymer liquid contains highly concentrated GM-tethered star-shape poly(AA) in water, which provides not only a large quantity of carboxyl groups for salt-bridge formations but also a substantial amount of carbon-carbon double bond for covalent crosslinks. In contrast, both Fuji II LC and Vitremer contain HEMA and/or other low MW methacrylate comonomers. The effect of aging on Example M, Fuji II, Fuji II LC and Vitremer on CS over a period of two weeks is shown in FIG. 10. As shown in FIG. 10, they have a lower strength as compared to Example M. Fuji II showed relatively higher CS but lower DT and FS as compared to Fuji II LC and Vitremer. Conventional CGICs do not produce any covalent crosslinks except for salt-bridges (ionic bonds) when they are set.

TABLE 7
YS, modulus, UCS in the course of aging.

| | | | | |
|---|---|---|---|---|
| Example | 1 h | 1 d | 1 w | 2 w |

YS¹ (MPa)

| | | | | | | | | |
|---|---|---|---|---|---|---|---|---|
| M⁴ | 81.7 | (0.9)^{a,3} | 202.3 | (7.2)^{c} | 274.1 | (2.1)^{A} | 278.5 | (11)^{A} |
| Fuji II | 95.1 | (0.8)^{b} | 199.9 | (2.7)^{c} | 200.1 | (3.0)^{B} | 204.2 | (10)^{B} |
| Fuji II LC | 87.8 | (2.3)^{a,b} | 120.9 | (10) | 125.7 | (7.0)^{C} | 141.5 | (9.4)^{C} |
| Vitremer | 32.2 | (2.4) | 87.3 | (0.7) | 104.9 | (5.8)^{D} | 101.7 | (7.3)^{D} |

Modulus (GPa)

| | | | | | | | | |
|---|---|---|---|---|---|---|---|---|
| M | 4.18 | (0.19) | 6.84 | (0.45) | 8.73 | (0.05)^{E} | 8.87 | (0.23)^{E} |
| Fuji II | 6.98 | (0.05) | 9.06 | (0.07) | 9.52 | (0.13)^{F} | 9.62 | (0.04)^{F} |
| Fuji II LC | 3.62 | (0.16) | 5.33 | (0.09)^{d,G} | 5.40 | (0.28)^{f,G} | 5.64 | (0.15)^{g} |
| Vitremer | 2.07 | (0.09) | 4.99 | (0.17)^{d} | 5.38 | (0.17)^{f} | 5.78 | (0.31)^{g} |

UCS² (MPa)

| | | | | | | | | |
|---|---|---|---|---|---|---|---|---|
| M | 217.5 | (4.0) | 272.9 | (8.5) | 334.9 | (5.4)^{H} | 335.2 | (4.5)^{H} |
| Fuji II | 152.0 | (0.3) | 235.6 | (4.4)^{e,I} | 252.0 | (7.4)^{I} | 251.4 | (6.9)^{I} |
| Fuji II LC | 181.5 | (12) | 212.7 | (15)^{e,J} | 219.1 | (1.7)^{J} | 208.6 | (11)^{J} |
| Vitremer | 88.9 | (4.5) | 148.1 | (0.6)^{K} | 153.5 | (2.3)^{K} | 150.8 | (1.6)^{K} |

¹YS = CS at yield;
²UCS = ultimate CS;
³Entries are mean values with standard deviations in parentheses and the mean values with the same superscript letter were not significantly different (p > 0.05);
⁴Grafting ratio = 50%, P/W ratio = 75/25 and P/L ratio = 2.7. Specimens were conditioned in distilled water at 37° C. prior to testing.

### Method Example

In vitro cytotoxicity. The in vitro cytotoxicity of Example M was studied using Balb/c 3T3 mouse fibroblast cells. It has been reported that RMGICs are more cytotoxic than CGICs (see, Leyhausen G, Abtahi M, Karbakhsch M, Sapotnick A, Geustsen W. “Biocompatibility of various light-curing and one conventional glass-lonomer cements” Biomaterials 19:559-564 (1998)). It has been suggested that certain leachable material, such as HEMA and incorporated photo-initiators and activators from RMGICs, which have been shown to cause adverse effects on cell viability and thus caused cytotoxicity (Geurtsen W, Spahl W, Leyhausen G. “Residual monomer/additive release and variability in cytotoxicity of light-curing glass-ionomer cements and compomers”. J Dent Res 1998; 77(12):2012-9), may be the cause. However, glass-ionomers generally are considered to be inert materials as compared to dental composite resins. Unpolymerized monomers my also be responsible for pulp cell cytotoxicity (Stanislawski L, Daniau X, Lauti A, Goldberg M. “Factors responsible for pulp cell cytotoxicity induced by resin-modified glass ionomer cements”. J Biomed Mater Res 1999; 48(3):277-88). RMGICs have been shown to cause the highest cytophatic effects on odontoblast cell line (MDPC-23) (de Souza Costa, Calif.; Hebling, J; Garcia-Godoy, F; Hanks, C T. “In vitro cytotoxicity of five glass-ionomer cements”. Biomaterials 2003; 24:3853-3858). The foregoing publications are incorporated herein by reference.

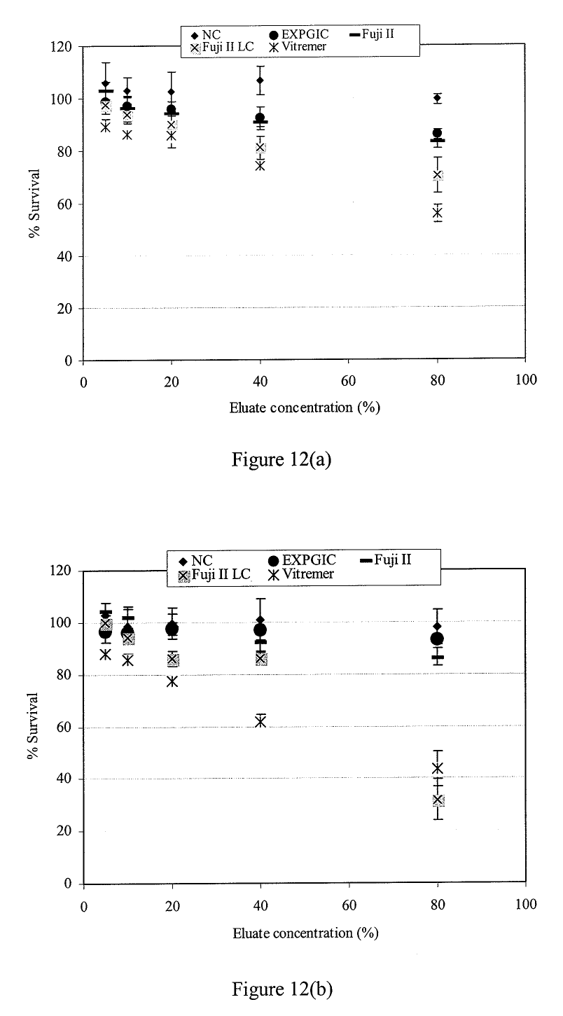

In vitro cell culture studies have been used as screening tests for evaluation of dental materials. Balb/c 3T3 mouse fibroblast cell lines were used to examine the in vitro cytotoxicity of Example M and compared it with those for commercial Fuji II, Fuji II LC and Vitremer, with the help of MTT assay. Example M was not expected to show any significant cytotoxicity and its in vitro cytotoxicity was expected to be as low as that of those CGICs because that example does not contain any comonomers in its formulation. FIG. 11 shows the cell viability after the cells were cultured with the eluates of Example M, Fuji II, Fuji II LC, Vitremer, and blank, i.e., negative control (NC). The viability (%) was in the decreasing order: (1) for the 3-day eluate, NC (99.4±1.9)>Example M (86.1±1.9)>Fuji II (83.4±2.6)>Fuji II LC (70.5±6.7)>Vitremer (55.8±3.2), where Example M and Fuji II were not significantly different from each other (p>0.05); (2) for the 7-day eluate, NC (98.1±6.7)>Example M (93.4±0.8)>Fuji 11 (86.1±3.3)>Vitremer (43.6±6.6)>Fuji II LC (31.7±7.8), where NC, Example M and Fuji II were not significantly different from each other (p>0.05). FIG. 12a and FIG. 12b show the cell viability vs. eluate concentration at the 3-day and 7-day extractions, respectively.

FIG. 11 shows the cell viability after the cells were cultured with the eluates of Example M, Fuji II, Fuji II LC, Vitremer, and blank, i.e., negative control (NC). The viability (%) was in the decreasing order: (1) for the 3-day eluate, NC (99.4±1.9)>Example M (86.1±1.9)>Fuji II (83.4±2.6)>Fuji II LC (70.5±6.7)>Vitremer (55.8±3.2), where Example M and Fuji II were not significantly different from each other (p>0.05); (2) for the 7-day eluate, NC (98.1±6.7)>Example M (93.4±0.8)>Fuji II (86.1±3.3)>Vitremer (43.6±6.6)>Fuji II LC (31.7±7.8), where NC, Example M and Fuji II were not significantly different from each other (p>0.05). FIGS. 12a and 12b show the cell viability vs. eluate concentration at the 3-day and 7-day extractions, respectively.

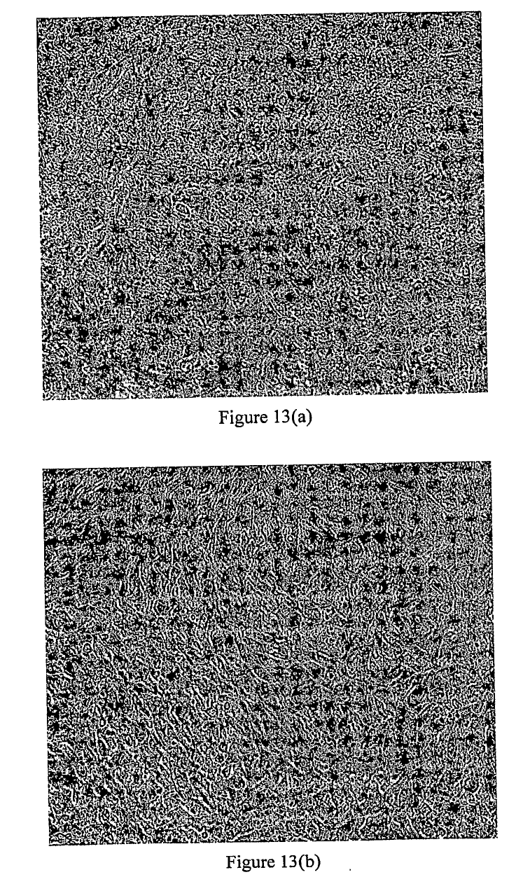

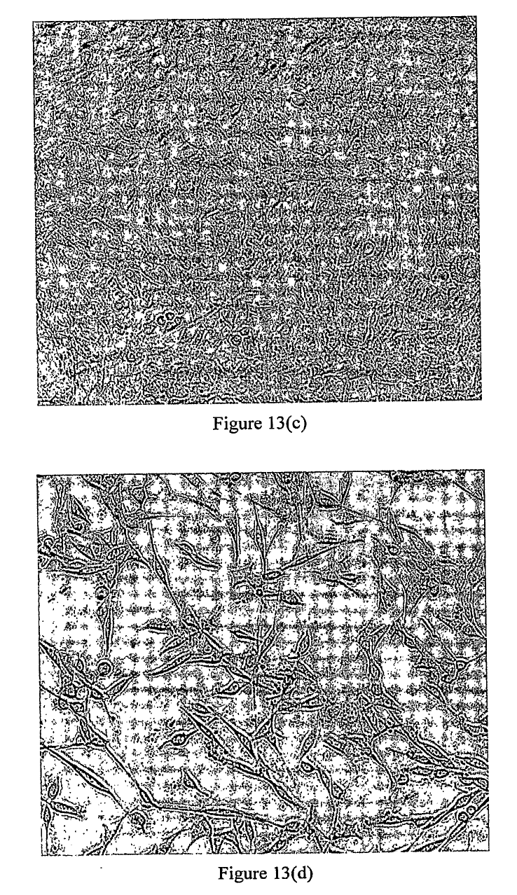

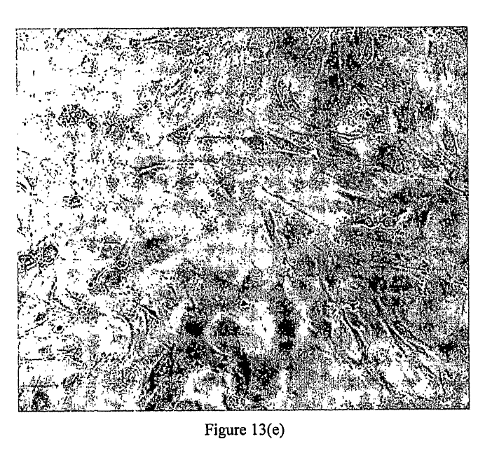

FIG. 13 is a set of optical photomicrographs describing the cell morphology and density after contact with the corresponding 7-day cement eluates. FIGS. 13a, 13b, 13c, 13d and 13e represent the cell morphology and density after cultured with NC, Example M, Fuji II, Fuji II LC and Vitremer.

From FIG. 11, except for NC, Example M showed the highest cell viability after cell exposure to both 3-day and 7-day eluates. Vitremer showed the lowest viability to the 3-day eluate whereas Fuji II LC showed the lowest viability to the 7-day eluate. This may be attributed to the fact that Example M contains no any comonomers before polymerization and thus no leachables (unreacted monomers) should be expected. Likewise, Fuji II showed very little cytotoxicity because it is a CGIC, which does not contain any leachable monomers or other additives such as photo-initiators and activators (Wilson A D, McLean JW. “Glass-ionomer cements”, Chicago, Ill.: Quintessence Publ Co.; 1988; Davidson C L, Mjör I A. “Advances in glass-ionomer cements”, Chicago, Ill.: Quintessence Publ Co.; 1999). Vitremer cement was reported to be the most cytotoxic among several tested cements including Fuji II LC (de Souza Costa Calif., Hebling J, Garcia-Godoy F, Hanks C T. “In vitro cytotoxicity of five glass-ionomer cements” Biomaterials 2003; 24:3853-3858; Stanislawski L, Daniau X, Lauti A, Goldberg M. “Factors responsible for pulp cell cytotoxicity induced by resin-modified glass ionomer cements” J Biomed Mater Res 1999; 48(3):277-88; Geurtsen W, Spahl W, Leyhausen G. “Residual monomer/additive release and variability in cytotoxicity of light-curing glass-ionomer cements and compomers” J Dent Res 1998; 77(12):2012-9), which has been attributed mainly to the photo-activator, diphenyliodonium chloride and partially to the comonomer, HEMA. In the case of Fuji II LC, it was believed that this cement is much less in vitro cytotoxic than Vitremer because there is no diphenyliodonium chloride in the formulation of Fuji II LC, although Fuji II LC contains HEMA (Geurtsen W, Spahl W, Leyhausen G. “Residual monomer/additive release and variability in cytotoxicity of light-curing glass-ionomer cements and compomers” J Dent Res 1998; 77(12):2012-9). However, the present study showed that Fuji II LC was more cytotoxic than Vitremer after the cells were cultured with the 7-day eluate, even though Vitremer showed a strong cytotoxicity to the cells for the 3-day eluate. This new finding suggests that the cytotoxic elutes from Fuji II LC may require more time to leach out of the specimens, as compared to the other LCGICs including Vitremer. Indeed, Fuji II LC was found to contain a substantial amount of HEMA in its liquid formulation by gas chromatography (Geurtsen W, Spahl W, Leyhausen G. “Residual monomer/additive release and variability in cytotoxicity of light-curing glass-ionomer cements and compomers” J Dent Res 1998; 77(12):2012-9). Additionally, the cytotoxicity of the materials was dose-dependent (see FIGS. 12a and 12b), see Stanislawski L, Daniau X, Lauti A, Goldberg M. Factors responsible for pulp cell cytotoxicity induced by resin-modified glass ionomer cements. J Biomed Mater Res 1999; 48(3):277-88. The eluate concentration at 80% showed the highest cytotoxicity. Regarding the cell morphology and density, it is clear that FIG. 13a (NC), FIG. 13b Example M and FIG. 13c (Fuji II) exhibit very high cell density and the cells ahnost grew full of a cell well. In contrast, there were a very few number of the cells in both cell wells containing Fuji II LC (FIG. 13d) and Vitremer (FIG. 13e), indicating that most cells died due to the cytotoxicity of the sample eluates. The cell morphology and density for the 3-day eluate were similar to those for the 7-day eluate.

The foregoing examples are set forth as illustrative embodiments of the invention described herein. However, it is to be understood that such examples are not to be construed as limiting the invention as otherwise described herein. Variations and combinations of the features described herein are contemplated. For example, such variations as applied to the invention are also found in the cited documents, the disclosures of which are incorporated herein by reference.

1.-22. (canceled)

23. A polymer core initiator of the formula: 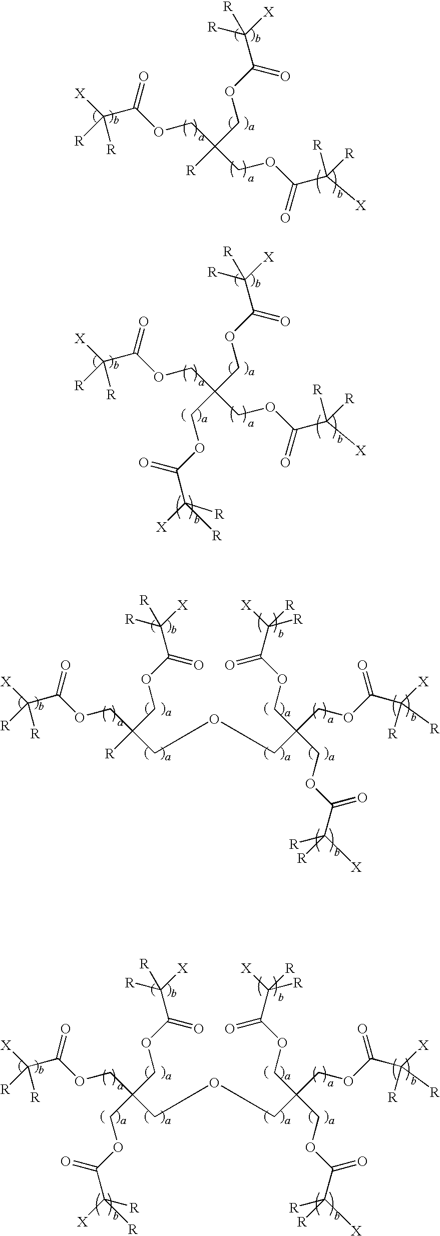
wherein in each instance R is hydrogen or an independently selected alkyl group; a is an independently selected integer from 1 to about 4; b is an independently selected integer from 1 to about 4; and X is a leaving group.

24. A polyfunctional prepolymer of the formula: 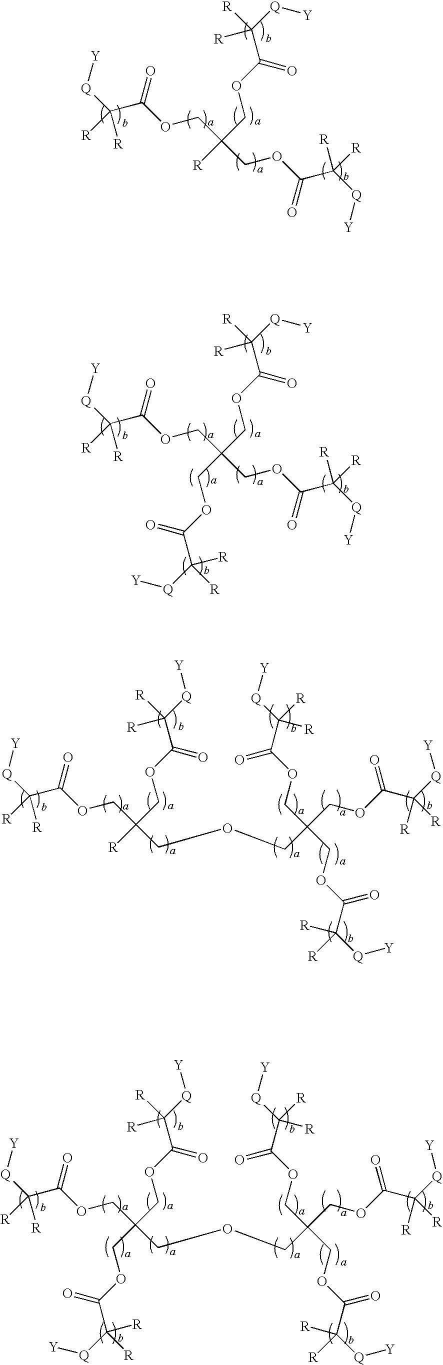
wherein in each instance R is hydrogen or an independently selected alkyl group; a is an independently selected integer from 1 to about 4; b is an independently selected integer from 1 to about 4; Q is an independently selected polymer of one or more acrylic acids, or ester, amide, or salt derivatives thereof; and Y is an independently selected leaving group.

25. The polyfunctional prepolymer of claim 24 wherein at least one of the acrylic acids forming the polymer Q is an ester or amide of an alcohol or amine each independently selected from the group consisting of acryloyloxyalkanols, acryloyloxyalkylamines, acryloylaminoalkanols, acryloylaminoalkylamines, each of which is optionally substituted, and combinations thereof.

26. The polyfunctional prepolymer of claim 25 wherein Y is halide.

27. The polyfunctional prepolymer of claim 24 wherein at least one of the acrylic acids forming the polymer Q is an ester of glycidyl methacrylate, an ester of 2-hydroxyethyl methacrylate, an amide of 2-isocyanatoethyl methacrylate, or a combination thereof.

28. The polyfunctional prepolymer of claim 24 wherein at least one of the acrylic acids forming the polymer Q is an ester of glycidyl methacrylate.

29. The polyfunctional prepolymer of claim 24 wherein at least one of the acrylic acids forming the polymer Q is amide of 2-isocyanatoethyl methacrylate.

30. A curable polymer composition comprising the polyfunctional prepolymer of claim 25, and an inorganic filler.

31. The curable polymer composition according to claim 30 wherein the inorganic filler is a fluoroaluminosilicate.

32. The curable polymer composition according to claim 30 wherein at least one of the acrylic acids forming the polymer Q is an ester of glycidyl methacrylate.

33. The curable polymer composition according to claim 30 wherein at least one of the acrylic acids forming the polymer Q is an amide of 2-isocyanatoethyl methacrylate.

34. The curable polymer composition according to claim 30 further comprising one or more acrylate co-monomers.

35. The curable polymer according to claim 33 further comprising one or more acrylate co-monomers selected from the group consisting of 2-hydroxyethyl methacrylate, methacryloyl beta-alanine, and combinations thereof.

36. The curable polymer composition according to claim 30 further comprising a redox initiator system.

37. A kit comprising the polyfunctional prepolymer of claim 25, an inorganic filler, and a container for preparing a curable polymer composition.

38. The kit according to claim 37 wherein the inorganic filler is a fluoroaluminosilicate.

39. The kit according to claim 37 wherein at least one of the acrylic acids forming the polymer Q is an ester of glycidyl methacrylate.

40. The kit according to claim 37 wherein at least one of the acrylic acids forming the polymer Q is an amide of 2-isocyanatoethyl methacrylate.

41. The kit according to claim 40 further comprising one or more acrylate co-monomers selected from the group consisting of 2-hydroxyethyl methacrylate, methacryloyl beta-alanine, and combinations thereof.

42. A method for repairing a defect in a mammalian tissue comprising the steps of placing the curable polymer composition of claim 30 in the defect, and curing the curable polymer composition.

43. The method of claim 42 wherein the curing step includes curing with radiation.

44. The method of claim 42 wherein the curable polymer composition further comprises a redox initiator system.

45. The method of claim 42 wherein the defect is a dental defect.

46. The method of claim 42 wherein the defect is a class I or class II cavity.
